(19) United States
(12) Patent Application Publication
(10) Pub. No.: US 2022/0401748 A1
(43) Pub. Date: Dec. 22, 2022

(54) COLD AIR PLASMA GENERATING APPARATUS

(71) Applicant: Bionic IP Pty Ltd, Gold Coast MC QLD (AU)

(72) Inventor: Deborah Louise Black, Robina Queensland (AU)

(21) Appl. No.: 17/650,704

(22) Filed: Feb. 11, 2022

(30) Foreign Application Priority Data
Jun. 18, 2021 (AU) 2021901835
Nov. 25, 2021 (AU) 2021903802

(51) Int. Cl. A61N 1/44; B01J 19/08

(52) U.S. Cl. CPC A61N 1/44; B01J 19/088; B01J 2219/0896; B01J 2219/0845

(57) ABSTRACT

An apparatus for generating cold air plasma includes a housing including a plurality of spaced openings; a plurality of needles, each needle being positioned in a respective opening; and a driver circuit that generates an electrical signal that is applied to the needles to generate a pulsed electromagnetic field (PEMF), wherein the electrical signal is alternating at a carrier frequency and is modulated at a pulse frequency to thereby generate cold air plasma. A method for providing therapy to a subject includes generating cold air plasma using an apparatus including: a) a housing including a plurality of spaced openings; b) a plurality of needles, each needle being positioned in a respective opening; and c) a driver circuit that generates an electrical signal that is applied to the needles to generate a pulsed electromagnetic field, wherein the electrical signal is alternating at a carrier frequency and is modulated at a pulse frequency to thereby generate cold air plasma.

### CROSS-REFERENCE TO RELATED APPLICATIONS

The Application claims the benefit of Australian Patent Application No. 2021901835, filed Jun. 18, 2021, and Australian Patent Application No. 2021903802, filed Nov. 25, 2021, which are incorporated herein by reference in their entirety.

### BACKGROUND OF THE INVENTION

The present invention relates to an apparatus and system for producing cold air plasma (CAP) reactive air species, optionally having a negative and/or neutral polarity, and optionally producing Hydrogen (H₂), and optionally generated using pulsed electromagnetic fields (PEMF) from ambient air temperature and non-equilibrium atmospheric air pressure.

### DESCRIPTION OF THE PRIOR ART

The reference in this specification to any prior publication (or information derived from it), or to any matter which is known, is not, and should not be taken as an acknowledgement or admission or any form of suggestion that the prior publication (or information derived from it) or known matter forms part of the common general knowledge in the field of endeavor to which this specification relates.

It is known that small negatively charged air ions are biologically active can lead to improved health outcomes through ingestion of negative air ions over time. It is known to provide ionizing devices capable of producing such negatively charged ions.

U.S. Pat. No. 5,973,905 describes a negative ion generator with a plurality of needle assemblies, each of which has a distinct needle end point. The generator includes a drive circuit for providing high voltages to the needle end points and a selection circuit by which the amount of and/or the frequency at which the ions are produced. The needle assemblies are replaceable by the user by way of plug and socket style connections. An indicating means is provided for indicating to the user of the generator the condition of the needle assemblies and when the needles should be replaced. The drive circuit can include a safety current route in the event of an earth fault.

### SUMMARY OF THE PRESENT INVENTION

In one broad form, an aspect of the present invention seeks to provide apparatus for generating cold air plasma, the apparatus including: a housing including a plurality of spaced openings; a plurality of needles, each needle being positioned in respective opening; a driver circuit that generates an electrical signal that is applied to the needles to generate a pulsed electromagnetic field (PEMF), wherein the electrical signal is alternating at a carrier frequency and is modulated at a pulse frequency to thereby generate cold air plasma.

In one embodiment the electrical signal and PEMF have varying frequency, intensity and multiple waveforms including at least one of: a non-square waveform; a right-angled waveform; a square waveform; a sine waveform; a triangular waveform; complex waveforms; Fourier modified waveforms; a sawtooth waveform; particle-wave duality; and, a sharks tooth waveform.

In one embodiment the carrier frequency is at least one of: in the range of 17 kHz to 18 kHz; in the range of 17.25 kHz to 17.75 kHz; and, in the range of 17.4 kHz to 17.6 kHz; or about 17.5 kHz±0.25%.

In one embodiment the pulse frequency is at least one of: in the range of 0.1 Hz to 40 Hz; in the range of 0.1 Hz to 4 Hz; less than 1 Hz; about 4 Hz; about 7.83 Hz; about 8 Hz; about 10 Hz; about 16 Hz; about 25 Hz; about 32 Hz; or about 40 Hz.

In one embodiment the apparatus includes a user input that allows selection of a pulse frequency and strength.

In one embodiment the driver circuit includes: a rectifier configured to be attached to a mains power supply; a switched mode power supply coupled to the rectifier; a pulse transformer coupled to the switched mode power supply; and a voltage multiplier that connects the pulse transformer to the needles.

In one embodiment the pulse transformer at least one of: is configured to generate an output voltage of about 3,500-4,500 volts and includes a 178:1 turns ratio.

In one embodiment the voltage multiplier is configured to generate an output voltage of about 17,500 volts.

In one embodiment the electrical signal at tips of the needles has a voltage at least one of: more than 12,000 volts; in the range of 12,000 volts to 14,000 volts; in the range of 12,500 volts to 13,500 volts; or about 13,000 volts±0.25%.

In one embodiment a power supply circuit includes a bridging resistor arrangement bridging an AC adaptor transformer.

In one embodiment the bridging resistor arrangement provides an earth reference via a neutral line of an alternating mains power supply.

In one embodiment the bridging resistor arrangement includes at least two resistors that provide two means of operator protection and a return path for the charged electrons from the circuit.

In one embodiment the apparatus is configured to provide a return current path allowing electrons stripped from molecules and/or ions to return to the device via the subject.

In one embodiment the apparatus includes an insulating member surrounding high voltage components.

In one embodiment the insulating member at least one of: has a convoluted shape; or provides a current flow path to the needles.

In one embodiment the apparatus includes an alternating current adaptor configured to attach to an alternating mains power supply.

In one embodiment the alternating current adaptor includes a barrel plug that connects to a barrel socket in the housing, and wherein the barrel socket is recessed within the housing to reduce electrostatic charge build-up.

In one embodiment the alternating current adaptor includes the pulse transformer.

In one embodiment the apparatus is configured to generate a corona cold air plasma and other quantum air species with a negative or neutral polarity at the tips of the needles and/or inside the plasma plume at the tips of the needles or just beyond the needle tips in a plume space of less than about 3 mm.

In one embodiment the apparatus includes a plurality of gold coated needle assemblies in electrical contact with a voltage multiplier, each needle assembly being configured to receive a respective needle.

In one embodiment the needles include: a shaft configured to be attached to a needle assembly within the housing; and a tip projecting into opening which is a hollow semi- circumference shaped opening.

In one embodiment the shaft is approximately 60% of the needle length.

In one embodiment the needles are made of a combination of metals.

In one embodiment the apparatus includes up to eight needles circumferentially spaced around a circular housing and where each four sets of needles are of equal distance from each other.

In one embodiment the apparatus includes one or more caps configured to be positioned in the openings to selectively deactivate one or more of the needles.

In one embodiment the apparatus is configured to generate at least one of and/or cause the generation of at least one of: reactive oxygen and nitrogen species (RONS) and/or their anti-particles; a complex mix of RONS and/or their anti-particles; reactive oxygen species (ROS) and/or their anti-particles; oxygen singlet, superoxides and/or their related and anti-particles; electrons (leptons), positrons, atoms and photons; muons, pions, protons, antiprotons (hadrons of quarks), neutrinos, anti-neutrinos, particle-wave duality, neutral quantum elements and quantum particles; force field energy quantum particles and waves; Ruach energy; Ruach life force; dark energy; antimatter; other quantum air species and quantum energy fields; REDOX effects inside and on the body; and, electroporation and/or plasmaporation on the skin.

In one embodiment the apparatus is configured to generate cold air species having an ion density for at least one of: more than 900,000 ions per cubic centimeter at one meter from the apparatus; more than 9.9 Million ions per cubic centimeter (9.9×10⁶) at 300 mm from the apparatus; more than 28 Million ions per cubic centimeter (28×10⁶) at 100 mm from the apparatus; and, more than 30 Million ions per cubic centimeter (30×10⁶) at 50 mm from the apparatus.

In one embodiment the apparatus is configured to generate cold air species with an ozone concentration of less than 0.04 ppm.

In one embodiment the apparatus is configured to generate air species with a flow rate for at least one of: more than 40.3 Trillion ions per second (40.3×10¹²) at 300 mm from the apparatus; more than 50.1 Trillion ions per second (50.1×10¹²) at 100 mm from the apparatus; and, more than 76.8 Trillion ions per second (76.8×10¹²) at 50 mm from the apparatus.

In one embodiment the apparatus is configured to at least one of: generate a pulsed electromagnetic field; generates ions in air proximate the housing; generates various air species including electrons and ions into air proximate the housing at high spinning atomic temperatures but cold to touch; generates various particle-wave duality air species including electrons and ions; transports ions away from the housing; transports various air species including electrons and ions into air portals away from the housing into or on the human; creates environment for collisions between PEMF and magnetic fields at right angles to generate cold fusion spark and photons of light energy inside the body; collisions through the circuit alters the field so that it points in a second direction perpendicular back to the apparatus; creates a biologically safe spark from cold fusion and produces photons of light energy, muons and pions inside the body; creates a biologically safe spark and photons of light energy inside the body that cause water electrolysis to take place to generate hydrogen H₂ in the body chemistry; leaves charged molecules inside the body whilst the electrical and magnetic current rips away electrons and/or neutrinos off the molecule and then out of the body; leaves charged molecules inside the body whilst electrons and neutrinos quantum tunnel out of the body away from the molecules and back into the circuit; pulls various air species including electrons and/or neutrinos back into the circuit and then to the created ground inside the housing; pulls electrons to ground and back into the apparatus where the electron returns to the needle and becomes a secondary electron holding a memory of its previous energy so it is more energetic than before being discharged back towards and into the subject; and, causes processes to occur repeatedly through the circuit at a magnetic speed close to the speed of light.

In one embodiment the apparatus is configured to provide a Selective Systemic Adaptogen therapy to a subject and response from the subject's body and/or creates the environment for homeostasis to occur within the subject.

In one embodiment the apparatus inactivates viruses by compromising the capsid using mechanical vibrations of the Air Species to surround and inactivate the capsid leaving its contents inactive.

In one embodiment at least one of: through the electric conductivity of the subject receiving the atomically hot, spinning and vibrating RONS and other quantum air species, resonates within the body's fluid systems after being stimulated by the Ruach life force field created by cold fusion collision of the fields within the circuit and the ripping of the electron off the ion creating photons at magnetic speed being close to the speed of light; and, through the electric conductivity of the subject receiving the atomically hot, spinning and vibrating negative oxygen containing ions from the CAP, combines with created photons at magnetic speed, from resonant frequency of the Air Species within the body's systems that are being carried throughout the fluid currents of the human body protected by water molecules before being stimulated by internal water electrolysis created inside the subject called the Ruach life forces.

In one embodiment the apparatus output frequency shapes are designed to mimic the geometrical shape of the SARS-CoV-2 virus for virus geometric and physics communication destruction and inactivation to achieve innate subject immunomodulates.

In one embodiment cold air gas plasma creates RONS and/or other air species which transfers reactivity into the liquids of the respiratory mucosal systems to induce further biochemical responses and pathogen inactivation as a result of the plasma and biofield communications through intra and inter-cellular water memory.

In one embodiment the electric conductivity in the subject is determined by the negative oxygen containing ions and not by the electrons which are ripped away from the ions leavingthe molecules inside the subject whilst the electron travels back into the circuit for ground potential.

In one embodiment, in use, a subject is positioned in a recommended manner appropriate for a therapeutic condition of at least one of: in the range 15 cm to 1 mtr from the housing; approximately 1 m from the housing; in the range of 1 m to 2 m from the housing; and, within a room containing the apparatus.

In one embodiment the apparatus is configured to drive air species and PEMF and resonance into the subject's respiratory system and/or skin and/or orifices at least in part by way of a common ground connection with the subject.

In one embodiment the apparatus is configured to restore aging of molecular and cellular pathogenesis by creating an environment of equilibrium and homeostasis through the combination of CAP, RONS, PEMF and an energy life force.

In one embodiment the cold air plasma and energy fields reduce the effects of the hormone systems abscisic acid which reduces the accumulation and potential of oncogenic stress, chromatin organization and gene expression of senescent cells, and in doing so protects the human from biological aging and reduces the gradual deterioration of functional characteristics in aging diseases.

In one embodiment the apparatus is configured to at least one of: provide a systemic selective adaptogen response; cause apoptosis; interact as a retrograde transport along nerves throughout the body particularly the vagus nerve; interact across the blood-brain barrier into the central nervous system; interact with the central nervous system to induce innate and adaptive immune responses throughout the body; induce immune responses by interacting with at least one of: Serotonin; Melatonin; Mitochondria; Hydrogen, hydrogen peroxide and/or hydroxyl radical, Lycopene; and Glutathione and Hormonal system and All body systems

In one embodiment the apparatus is configured to treat and prevent any one of or combination of the following as a systemic selective adaptogen solution: respiratory conditions; asthma and bronchitis; chronic obstructive pulmonary disease (COPD); emphysema; anosmia; silicosis; lung diseases; black lung disease; SARS-CoV-2 (COVID-19) including all mutations; Common colds and influenza including all mutations; All viral, bacterial and other pathogen infections and mutations; All viral, bacterial and other pathogen long haul symptoms and infections; Sleep apnoea; cancers; basal cell carcinoma; breast cancer; glioblastoma; brain cancers; lung cancers; bladder cancer; endometrial cancer; metastasized cancers; regulate inflammation; stimulate a selective systemic regulatory immune response; diabetes; depression and PTSD; anti-ageing; Alzheimer and vascular dementia; reduce blood pressure; skin conditions; psorisis; eczema; dermatitis; diabetic foot wounds; surgical wounds; burns and scarring; auto-immune diseases; Lupus; Hashimoto's; Transverse Myelitis; Fibromyalgia; Diabetes; Lyme disease; Ross river fever; Brain fog; and, All allergies.

In one broad form, an aspect of the present invention seeks to provide a method for providing therapy to a subject, the method including generating cold air plasma using an apparatus including: a housing including a plurality of spaced openings; a plurality of needles, each needle being positioned in respective opening; and, a driver circuit that generates an electrical signal that is applied to the needles to generate a pulsed electromagnetic field, wherein the electrical signal is alternating at a carrier frequency and is modulated at a pulse frequency to thereby generate cold air plasma.

In one broad form, an aspect of the present invention seeks to provide apparatus for generating corona cold air plasma, the apparatus including: a housing including a plurality of spaced openings; a plurality of needles, each needle being positioned in respective opening; a driver circuit that generates an electrical signal that is applied to the needles to generate a pulsedelectromagnetic field (PEMF), wherein the electrical signal is alternating at a carrier frequency and is modulated at a pulse frequency to thereby generate corona cold air plasma RONS and other quantum species of negative and neutral polarity energized by Ruach life force, and wherein when a subject is exposed to the generated corona cold air plasma, the apparatus induces a systemic selective adaptogen response inside the subject.

In one embodiment the systemic selective adaptogen response is induced and energized by Ruach life force field energy and its subsequent water electrolysis for Hydrogen (H₂) production.

It will be appreciated that the broad forms of the invention and their respective features can be used in conjunction and/or independently, and reference to separate broad forms is not intended to be limiting. Furthermore, it will be appreciated that features of the method can be performed using the system or apparatus and that features of the system or apparatus can be implemented using the method.

### BRIEF DESCRIPTION OF THE DRAWINGS

Various examples and embodiments of the present invention will now be described with reference to the accompanying drawings, in which:

FIGS. 1 and 2 are rear and front elevational views illustrating an example of a negative ion generator;

FIG. 3 is a cut-away perspective view illustrating a needle assembly for the negative ion generator apparatus of FIG. 1;

FIG. 4 is an enlarged view of a second terminal connector means being part of the assembly of FIG. 3;

FIG. 5 is a schematic block diagram of the negative ion generator of FIG. 1;

FIG. 6 is a schematic diagram illustrating the control and operating circuitry of the negative ion generator of FIG. 1;

FIG. 7 is a schematic diagram illustrating the voltage multiplier circuit used by the negative ion generator of FIG. 1;

FIG. 8 is an example driver circuit for the negative ion generator FIG. 1;

FIG. 9 is a schematic diagram of an example of a power supply circuit for a cold air plasma generating apparatus;

FIGS. 10A and 10B are images of a Basal Cell Carcinoma on the back of the right ear of a patient prior to treatment;

FIGS. 10C and 10D are images of the Basal Cell Carcinoma of FIGS. 10A and 10B after treatment using the cold air plasma generating apparatus; and,

FIGS. 11A to 11D are images of an acrotic heel undergoing treatment using the apparatus at zero weeks, three weeks, four weeks and twelve weeks, respectively;

FIG. 12A is a schematic diagram of an example of operation of a traditional negative ion generator and the subject circuits;

FIG. 12B is a schematic diagram of an example of operation of the subject circuits used by this cold air plasma generating apparatus;

FIG. 13A is an image of an example of a deliberate crack in the housing showing evidence of electron pathways;

FIG. 13B is a schematic diagram of an example of an insulting member;

FIGS. 13C and 13D are three dimensional renderings of an example of an insulating member; and,

FIGS. 13E and 13F are images of an example of an insulating member within a cold air plasma generating apparatus.

### DETAILED DESCRIPTION OF THE PREFERRED EMBODIMENTS

An example of an apparatus for generating only negative ions will now be described.

In the current example, the apparatus includes a housing including a plurality of spaced openings, and a plurality of needles, each needle being positioned in respective opening. In one example, the openings and needles are circumferentially spaced within a housing having a circular shape in plain view, although it will be appreciated that other arrangements could be used.

The apparatus further includes a driver circuit that generates an electrical signal that is applied to the needles to generate a pulsed electromagnetic field (PEMF), wherein the electrical signal is alternating at a carrier frequency and is modulated at a pulse frequency to thereby generate negative ions.

In one example, the physical construction of the apparatus is broadly similar to that of the negative air ion generator described in U.S. Pat. No. 5,973,905, and an example of this predecessor arrangement will now be described.

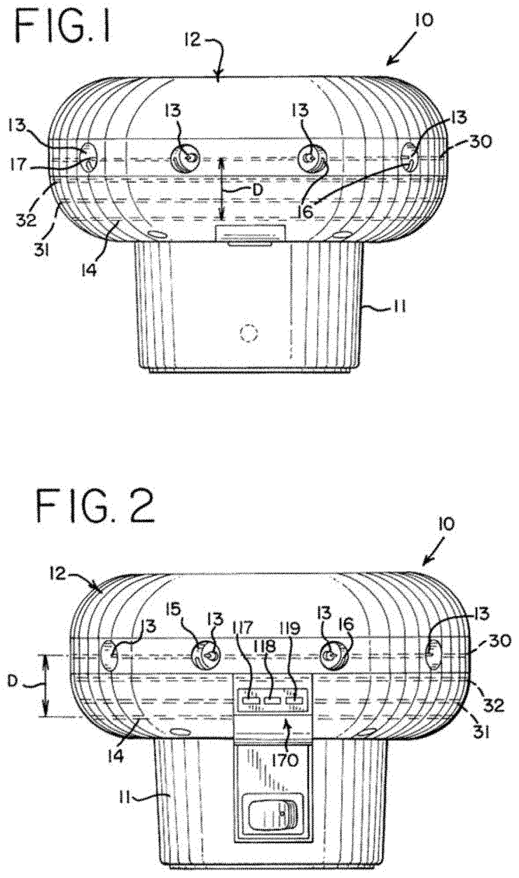

Referring to the drawings and initially to FIGS. 1 and 2, there is illustrated a negative air ion generator 10 which is generally mushroom shaped having narrow cylindrical body portion 11 and an upper enlarged cylindrical body portion 12. The negative air ion generator 10 is provided with a circular array of needle emitters 13 with eight in total and four being disposed generally in opposite directions on the upper body portion 12. Located at a separation distance D of approximately 17 mm below the ring of emitters 13 is an earth ring 14 shown in phantom and this is positioned from 15 mm to 20 mm with 17 mm as shown being optimum for the generation of small ions. Each emitter is located in a recess 15, each recess having as lot 16 for flow of ions between the needle points 17 and the earth ring 14.

As can be seen, the negative ion generator is relatively compact when compared with prior art devices and includes a high voltage circuit on a board 30 and a control circuit 31 which are separated within the unit using an insulating disc 32 of 1 mm thick polycarbonate is disposed between the boards 30 and 31.

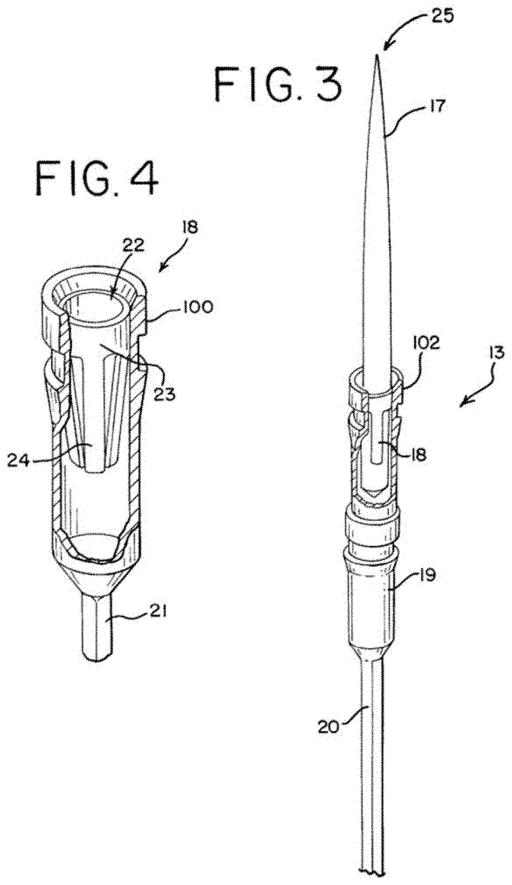

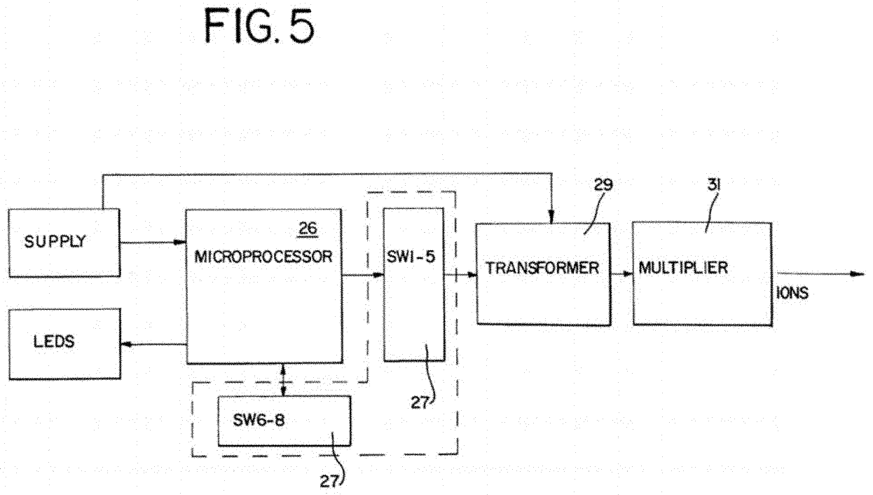

Referring now to FIGS. 3 and 4, there is illustrated a needle emitter assembly 13 having a replaceable needle point 17 releasably retained in a second connector 18 shown in cut-away. The second connector is also releasably retained in the first socket of a first connector 19, the first connector includes a pin 20 adapted to be soldered in circuit to provide fixed connector having a socket configured internally in similar fashion to the socket illustrated in cut-away in FIG. 4. Both the connectors 18 and 19 are configured with the same general internal construction, having a plug 21 and a socket assembly 22, the socket assembly 22 having an internal sleeve 23 with resiliently biased legs 24 adapted to frictionally engage the needle point 17 as shown in FIG. 3, in case of connector 19 the plug 21 of the connector 18 when inserted into the socket of the connector 19.

In this construction, the replaceable needle 17 is held within a second connector (FIG.4) 18 having a socket assembly 22 that includes an inner sleeve 23 that is held within a second housing portion 100 of the second connector 18, such that the inner sleeve 23 and the housing cooperatively define a second socket that receives the replaceable needle 17. As shown in FIG. 3, the socket assembly 22 and particularly the second housing portion 100 of the second connector 18 has a plug end 21 that is received within a first housing portion 102 of the first connector 19 that a first socket of the overall needle assembly 13 and that is insertable into the ion generator housing.

In the illustrated embodiment, all of the surfaces of the connector 19 and the connector 18 are gold plated to minimize corrosion, bearing in mind any shoulder, edge or defect, for example, arising due to corrosion can provide a site for the generation of ions and thereby reduction in the overall life of the needle assembly. In addition to the aforementioned gold plating, the needle may itself be formed from a corrosion resistant material, such as a rutheniumalloy. The coating and/or needles can include different densities.

It will be appreciated that the optimum is for ions to be generated at the very tip 25 of the needle point 17 rather than at other positions on the needle assembly. Use of the corrosion resistant coating enhances the production of ions at the needle point and prolongs the life of the needle assembly.

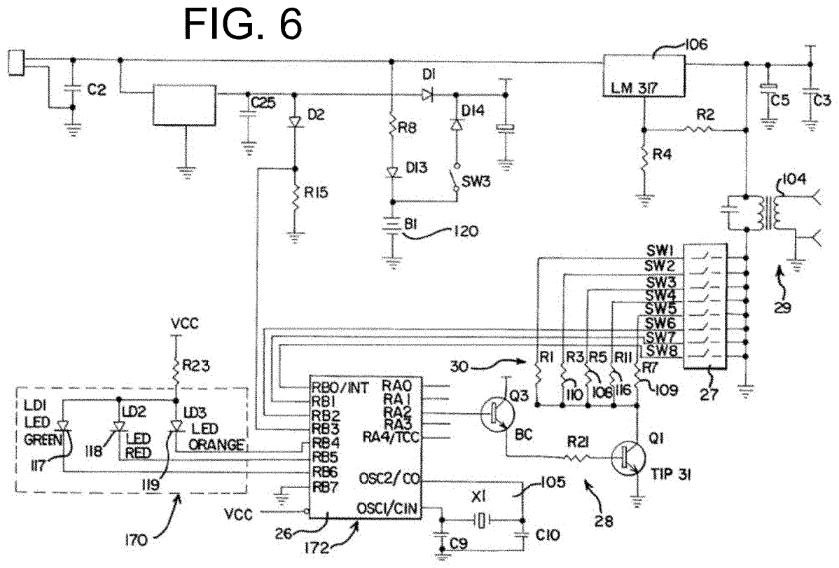

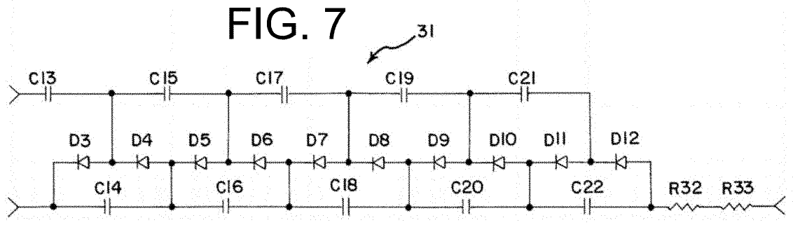

In one example, the negative air ion generator uses a Cockcroft Walton multiplier illustrated generally at 31 in FIG. 7, to generate high tension voltages of between 5 and 13 kV, derived from a ferrite tuned transformer 104 in FIG. 6 driven at 17 kHz. This provides a much more stable and medically effective output than in prior devices.

In present unit, the main supply is switch mode regulated and transformed to give 12 volts DC via a customized AC mains adaptor, which is then regulated to 10 volts DC for driving the main control circuitry. This contains a microprocessor 26 which is crystal controlled by a crystal oscillator X1, illustrated generally as 105 in FIG. 6. This in turn is modulated under control of switches 6 to 8 on DIP (dual in-line package) switch 27. The pulses are fed to a transistor circuit 28 that feeds to transistor Q1, first and then to transistor Q3, as shown in FIG. 6 which energizes the pulse transformer 29 capacitively tuned to act in class C mode. The drive to this is limited by a set of resistors at 30 selectable by switches 1 to 5 at DIP switch 27 to give a range of ion outputs. The transformer 29 is used to generate an output voltage which is then multiplied by a five-stage multiplier 31 shown in FIG. 7 to generate high voltages necessary for ionization.

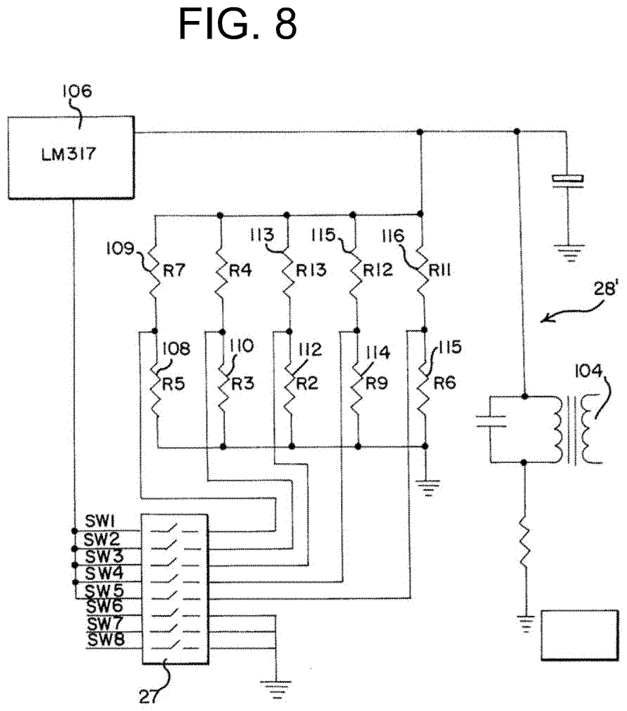

An alternate arrangement 28′ to the Darlington driver circuit 28 is shown in FIG. 8. In this arrangement, the coil resistance of resistor R4 is held constant at 1 ohm and a set of switches SW1 to SW5 change the settings on the variable voltage regulator LM317(106). The settings are changed by switching in different ratios, the resistor sets R5, R7, R3; R2, R13; R9, R12 and R6, R11, illustrated respectively as 108, 109, 110, 112, 113, 114, 115 and 116. This stratagem permits the inductance of the transformer 104 to vary and, with switching two ratio sets in parallel, the ionizer can give 9 output voltages from 5 switches of the DIP switch 27.

The modulation switches the pulse train on and off at a rate controlled by switches 6 to 8 of the DIP switch 27. Thus, it can be seen that the ion generation signal may be frequency modulated with the modulation frequency chosen from a range of frequencies and with the modulation frequency being selectable by changing the settings of the DIP switch 27, and in particular the settings of switch numbers 6-8 thereof.

However, the existing arrangement includes a number of modifications to the device of U.S. Pat. No. 5,973,905, leading to improved functionality. In one particular example, this allows the apparatus to be used to treat a biological subject, specifically to provide a ‘Systemic Selective Adaptogen Response’ so that the apparatus can be used in treating a wide range of different medical or other conditions.

Such benefits can be achieved by having the apparatus generate the pulsed electromagnetic field so as to generate cold air plasma species, and in one example, quantum cold air plasma species, including but not limited to reactive oxygen and nitrogen species (RONS), reactive oxygen species (ROS), oxygen singlet, and complex quantum and atomic air species thereof of a negative and/or a neutral polarity and/or anti-particles thereof moving at speed with fast spin and atomic heat but are cold to touch. In one particular example, the voltage power is required to be stronger without generating ozone to produce the oxygen singlet which is a preferred antioxidant neutral air species because it transfers its energy to molecules and macromolecules exciting them and supporting macrophage production of the same before it returns to its ground state. This combination is one particular example of a selective adaptogen process. Then the suitable configuration of the amplitude, shape and spin of the PEMF in the circuit creates a complex portal to transport all air species fast and safely to the subject for biological benefits.

In one example, the Air Species combine with the energy from the PEMF through the circuit to interact with the central nervous system (CNS) via the vagus nerve within the respiratory system as a retrograde transport, providing the CAP species with direct access to cross the blood brain barrier (BBB), and allowing the species to induce an effect within and using the subject's own hormonal and immune responses as directed from the CNS specific to the subject's own biological needs being another example of the selective adaptogen function using the subjects own systems.

In one particular example, the apparatus is used by placing the apparatus proximate to the subject when they are sleeping, for example by placing the apparatus within 1 m of the subject. As a result, the effectiveness of the arrangement is enhanced because blood serotonin levels naturally fall whilst sleeping, which causes protective blood Melatonin levels to rise. This ensures the CAP apoptosis caused by the species to damage adverse cells and elements induces a natural immune response providing a corresponding protective mechanism specific to that subject's needs and determined by the subject's own biology.

Extensive testing has shown no adverse health events and no recalls, and a range of different positive biological health outcomes, discussed in more detail below, that can be obtained repeatably and consistently.

To facilitate this process, and in contrast to the teaching of U.S. Pat. No. 5,973,905, the apparatus uses an electrical signal that has a mix of waveforms to create what is named ‘portals’, and in particular includes one or more complex waveforms, such as but not limited to Fourier modified waveforms, sawtooth waveforms, and in one preferred example, a sharks tooth waveform. Such waveforms result in complex pulsed electromagnetic fields, which facilitates transport of CAP from the needle tip, and causes the CAP species to diffuse into the body via the air/mucous interchange of the respiratory system, and/or into the skin and/in and around the entire body through a transdermal application using electroporation and/or plasmaporation. Additionally, the use of additional and multiple concurrent wave combinations create the ‘portals’ and can help bring the air species back onto the required waveform for the subject if interference exists.

In one example, the carrier frequency is in the range of 17 kHz to 18 kHz, 17.25 kHz to 17.75 kHz, or 17.4 kHz to 17.6 kHz, and in one example, preferably has a frequency of about 17.5 kHz±0.25%. It has been found that by more tightly constraining the carrier frequency in this manner provides for greater control over specific medical treatments, which can be tailored based on the pulse frequency and other variables used.

The pulse frequency is typically in the range of 0.1 Hz to 40 Hz, which notably expands on the ranges described in U.S. Pat. No. 5,973,905, by allowing for pulsing in the range of 0.1 Hz to 4 Hz. In one example, the device is configured to allow pulsing at different set frequencies, including any one of: more or less than about 0.1 Hz; about 0.5 Hz; about 1 Hz; about 2.5 Hz; about 4 Hz; about 7.83 Hz; about 8 Hz; about 10 Hz; about 16 Hz; about 25 Hz; about 32 Hz; or about 40 Hz. The pulse frequency signal can also have different signal magnitudes, resulting in different intensity of treatment and/or strength effects. To achieve this a user input is provided, such as one or more input buttons, which allow selection of a desired pulse frequency and/or magnitude, allowing different treatment options to be implemented.

The frequencies are selected so that the subject and apparatus or illness and apparatus interact as a “tuned system” of two oscillators having identical resonance frequencies. The Air species oscillate as a result of the PEMF, circuit and mechanical design, so that the body of the subject is activated by the signal close to the same resonance, which in turn enhances the effect of the treatment, by maximizing the resulting biological reactions induced by the Air Species and the circuit energies.

In the current example, the driver circuit includes a rectifier configured to be attached to a mains power supply, a switched mode power supply coupled to the rectifier, a pulse transformer coupled to the switched mode power supply and a voltage multiplier connecting the pulse transformer to the needles.

The pulse transformer has a 178:1 turns ratio and is configured to generate an output voltage of between 3,500-4,500 volts AC peak-to-peak. This is multiplied by the multi-stage voltage multiplier, which is configured to generate an output voltage of up to 13,000 volts, which is applied to the needles, resulting in an electrical signal at the needle tips that has a voltage that is more than 12,000 volts, and is typically in the range of 12,000 volts to 14,000 volts, or 12,500 volts to 13,500 volts, and which is preferably about 12,500 volts±2%.

In comparison to the arrangement of the device's predecessor in U.S. 5,973,905, the apparatus uses a different transformer configuration to increase the initial voltage and reduce tolerances, so that the resulting PEMF has a greater magnitude at a more accurately controlled frequency with a stronger current. This allows for improved transdermal penetration and a higher generation of cold air plasma formed Air Species at the needles, whilst controlling the output to reduce and/or prevent production of harmful ozone, in turn allowing the apparatus to be now introduce placement theory utility as another method of subject treatment due to more variable distances impacting strength of application to the subject.

In one example, the driver circuit includes a bridging resistor arrangement bridging the AC mains adaptor transformer, which provides an earth reference via a neutral line of an alternating mains power supply. The transformer used in the AC mains adaptor is also a pulse transformer. In one particular example, the arrangement of the pulse transformer, as well as the rectifier and switched mode power supply are provided in an alternating current mains adaptor, and an example of this is shown in FIG. 9.

In this example, the AC mains adapter includes international plug connections in the form of live and neutral plug pins, that are configured to be inserted into a main electrical socket. The AC adaptor delivers regulated DC power via a barrel plug, which connects to a barrel socket in the housing, which is recessed within the housing to reduce electrostatic charge build-up. The DC supply is thus referenced to AC mains ground via the aforementioned bridging resistors, providing a return path for electrons emitted from the voltage multiplier for the generation of ions. The return current path allows electrons stripped from ions and/or molecules to return to the device via the subject, which can enable enhanced therapeutic benefits as will be described in more detail below. For example, this could occur as a result of electrons being stripped from ions at, during and/or after the creation of RONS, and/or could be as a result of electrons being stripped from ions within the body leaving molecules. In one example, the current path is via the ground connection in combination for example with a conductive grounding mat on the bed or chair, although this is not always essential, and alternatively an additional return path could be provided by using a physical lead connection between the subject and the apparatus, for example using a conductive wristband or similar connected to the electrical ground of the apparatus.

The pins 951.1, 951.2 are connected to a rectifier 953, with the live pin 951.1 being connected via a fuse. Outputs from the rectifier are connected in series to the switched mode power supply 954 and inputs of the pulse transformer 955, which causes a switched DC current to flow through the transformer. Outputs of the pulse transformer 955 are connected to a regulator 956 and connectors 957.1, 957.2 of a barrel plug, which connects to a barrel socket in the housing, which is recessed within the housing to reduce electrostatic charge build-up. The output from the transformer is a regulated AC signal having a maximum supply current of about 120 mA at 13 KV and 40 Hz, with this being provided to the internal componentry within the housing, including the voltage multiplier.

The bridging resistor arrangement includes two or more resistors in series, which include a total resistance of approximately 20 MΩ. This also acts as a safety mechanism in event that the mains ground terminal is faulty, preventing hazardous current flow from the AC mains through to the DC supply for the product, so that two or more means of operator protection are used. Additionally, the bridging arrangement provides a ground current path via the neutral line of the mains electrical supply, assuming the mains system is earth referenced by the neutral line, which in practice provides a common ground with the subject and a novel circuit. The circuit means that the desired small extremely fast short- and long-lived species reach the mucous membranes without the generation of harmful ozone and without pathway collisions with air nuclei and water molecules in the ambient air which would slow it down. The PEMF current facilitates and pushes the transfer of the Air species into the subject, a push and pull transfer of energy through the circuit leaves a consequential initiation of Ruach life forces at the junction of these actions inside the subject. Between the PEMF push and the circuit magnetic pull the collision takes place where the molecule stays within the subject and is energised by the Ruach life force field caused by the electron being ripped away suddenly and quantum tunnelling out of the body flying back into the perpendicular circuit. This Ruach life force field created between the push and pull of the circuit collisions generates photons of light inside the body for new molecular interactions being created within the body and the production of photon wave frequencies as a storage of energy protected by water molecules inside the body. Specifically, electrons and neutrinos are attracted to ground via the subject from the circuit pull forces which causes them to travel through the subject as they search for ground at magnetic speed. This cascade of events helps provide the systemic selective adaptogen biological processes to safely initiate and complete using the subjects now energized biological intelligence immediately and for up to some hours. Thus, the spinning hot ions enter the body's biology with the spare electron and then there is a fusion event caused by a sudden ripping removal of the electron off that ion and making it now just a molecule which remains within the body. The event causes a fusion episode, which generates a spark of light (photon). This Ruach life force is an energy and when it takes place it creates a cell voltage which is enough for Hydrogen (H₂) to be created from the water electrolysis caused. That energy is then in the body protected by water molecules for some hours, whilst the electron returns to the apparatus ground generating a secondary electron.

As previously mentioned, the apparatus is configured to generate air species at the needle tips, typically through a type of corona CAP discharge plume, which occurs when high voltage is applied to create PEMF strong enough to create chain reactions of electrons colliding with atoms hard enough to ionize them which creates more free electrons to ionise more atoms causing the CAP RONS Air Species at the needle tips and within mm inside the discharge plume. To achieve this, the apparatus typically includes a plurality of gold coated needle assemblies in electrical contact with a voltage multiplier, each needle assembly being configured to receive a respective needle. The needles include a shaft configured to be attached to a needle assembly within the housing and a tip projecting into an opening in the housing. The needles are circumferentially spaced around the device, with each needle producing plasma. The housing is constructed of outgassing resistant medical grade plastics, to provide additional protection for allergy sufferers, whilst adding robust quality and high voltage protection.

The shaft of the needle is now approximately 60% of the needle length, which helps assist in improving the generation of a much higher output of the electrons in order to create the quantum species inside the plasma plume at the needle tips. In particular, compared to the needles of U.S. Pat. No. 5,973,905, the needles have thicker shaft and head to carry larger voltage across an extra 4.28 mm span on the needle length itself. The tapering now begins at 60% (4.28 mm)of the needle head, which increases the surface area for the electron energy up to the needle tips to assist in the air species generation, without increasing the ozone production.

The needles are typically made of a mix of corrosive resistant ROHS (Restriction of Hazardous Substances) compliant metals, treated to ensure smoothness and sharp consistency, whilst the needle assembly uses a thicker and denser gold layer with a more refined depth and tolerance and a higher quality gold, allowing for a lower resistance connection, which in turn allows a higher needle tip voltage, which improves generation of the Air Species, without generating more ozone.

In this regard, the original apparatus was typically only configured to generate 50,000 to 400,000 negative ions per cubic centimeter at ‘one meter’ from the apparatus. This new and improved design now typically generates more than about 900,000 negative ions per cubic centimeter at the same ‘one meter’ distance from the apparatus, thereby significantly altering the device air species from just ‘negative air ions’ to now ‘CAP Air species and other quantum Air Species with both a negative and neutral polarity’ for improved variable treatments.

The ability to generate such a high rate of electrons and other quantum air species inside the CAP for travel at magnetic speed (speed of light) through the circuit is particularly beneficial to reach the human biology for example pathogen destruction and immune protection. For example, the excited state of Oxygen O₂, is a very strong oxidant but not a radical called oxygen singlet in its quantum state ¹O₂ or O. It acts like a neutrino. It has all its electrons spin paired so it is charge neutral and its rate of decay is slow (54-84 milliseconds) despite being kinetically unstable in ambient air. It is paramagnetic due to its net orbital flips (not electron spins) and electronic angular momentum as seen by its electron paramagnetic resonance, energizing the formation of internal magnetic fields because it is attracted by the magnetic field which is at right angles to the electrical field so its travel in the portal circuit is above and below the electrical field.

O₂, water, calcium, blood and fat for example are weakly diamagnetic so are subtly repelled by the magnetic field. The human body is predominantly water and diamagnetic, when the human is grounded to earth with the apparatus as in this case the PEMF and strong electromagnetic current applied induces a strong diamagnetism and causes the creation of a magnetic field that then opposes the external field thereby returning particle-wave duality electrons through the circuit portal after the superconductor (being the human earth ground potential in this case) acted as the strong diamagnet source fed by the power of the electrical circuit. So as the magnetic field is applied to the body it creates an induced magnetic field in the opposite direction causing the repulsive force entirely from its interior and hence part of the pulling action which suddenly rips the electron away from the molecules in the body to quantum tunnel back to the portal circuit for the grounding ring destination inside the device.

¹O₂ is the primary agent of photooxidative stress in microorganisms reacting with a large range of macromolecules including proteins, lipids, DNA and RNA thereby forming further substances like organic peroxides and sulfoxides. It is indispensable in mitochondrial electron transport, fatty acid oxidation and vitamin metabolism. Microorganisms that face high light intensities such as carotenoids have protective mechanisms which react with ¹O₂ to prevent further damage of cellular functions thereby balancing through Systemic Selective Adaptogen activities. Further because the electron spin is zero in this case, this leads to its destructive behaviour on respiratory pathogens like viruses, whilst on the other hand its impact with the aqueous solution of the upper respiratory system produces the body's own hydrogen peroxide H₂O₂, to selectively complete the inhibitory and bactericidal effects on the upper respiratory tract whilst decomposing and regulating the ¹O₂ from further destruction. Hence, the body self regulates at its own pace.

To appreciate the importance of size, speed in the atomic and molecular world, the approximate width of the oxygen singlet ¹O₂ is 0.00014 μm (0.140 nm), negative ion of oxygen O₂ is 0.00012 μm (0.1 nm) whilst the free flowing energy electron is significantly less in size at 5.64×10⁻¹⁷ μm or 0.00000000000000564. When generated at the needle tips inside the cold air plasma plume the air species are cold to touch but hot atomically as they are produced under high energy, high forces, high flow rates and gain fast spin and subsequent atomic heat.

The PEMF and electromagnetic currents in the portals protect the Oxygen singlet and other quantum air species as they travel into the human biology where they are magnetically attracted to the foreign virus capsid which is an opposite charge and can destroy it through sheer large quantity of air species with vibrational heat, resonance, frequency mirroring and energy impacts. For example, this mechanical physics disruption has resulted in successful treatment against the SARS-CoV-2 enveloped Icosahedral capsid symmetry which is a much larger size of about 0.12 μm, so a significant number of the Air Species this apparatus produces being significantly smaller about 0.000000000000000564 μm shows it is able to easily penetrate the lipid barrier and spike proteins to surround all 20 triangles of each capsid with their vibrating heat and with the combined Ruach life force energy collisions and photons of light created are able to destroy and crack a capsid, effectively blowing it up with mechanical physics forces and rendering its content inactive. Furthermore, as the CAP is selective, any damage to the cells are rectified by the subjects own initiated immune, redox and other natural biological responses. Lastly the subjects cell membranes, inter and intra cellular fluids hold a memory of all these reactions. This stored memory has shown its ability to protect the subject from future pathogen infections either in their entirety or through reducing their severity. Accordingly, being able to generate up to 28 Million ions/cc at a flow rate of 50.1 Trillion ions/second at a 300 mm distance and 900,000 ions/cc at the one metre distance without any higher levels of ozone allows for improved treatment in more subjects in this biologically active range as a Systemic Selective Adaptogen.

The Air Species fly out of the CAP plume from the needle tips, being pushed through the PEMF portals driven by the PEMF to the subject and then pulled by circuit forces after the collisions inside the subject which created the Ruach life force fields in the process as the electrons are detached from the molecules to enter the portal circuit again in a magnetic attraction to seek the lowest potential they can find, which is anything connected to the electrical mains system, including the actual earth. When the air species collide with a ground connected conductive surface (the conductive grounded human being is that path in this case), they lose their spare electron into the current to flow through the portal to the ground path. The circuit is completed via the special isolation bridge modification in the AC mains adaptor, the electrons then find their way back into the apparatus grounding ring via the gap under the needle configuration, where they are raised up to high potential, and are ejected again into the air to form more CAP air species. As this novel circuit is self-fulfilling, the air species flow at magnetic speed from the device—through the human—and back into the device to complete the circuit, generating a gentle ion breeze or ion wind without a motor. The air species is therefore moving at what we know today as magnetic speed (close to the speed of light) as the polarity causes the electro-magnetic pull back to the lowest potential. A complex process that brings a multitude of active mechanisms biologically too numerous to detail but can be summarised as assisting the body reach homeostasis as a ‘systemic selective adaptogen’.

It is theoretically shown that elementary particle's such as Muons are also involved as a result of this novel circuit cycle in a different mechanism and assist the safe forces and charged particles of energy inside the human body. Muons are created when high energy protons collide with atomic nuclei of molecules and produce pions the strong force interactions which hold the nucleus together (Ruach life force). The muons carry the energy and momentum which react with the PEMF and the pulling force fields of the circuit ground creating the high energetic photons. This forcefield causes a collision of push and pull effects through the body and strips the electrons (leptons) from molecules and/or hydrogen atoms permitting the protons to pass through the body, the protons continue acceleration until they collide with antiprotons (hadrons made of quarks) in the circuit. Muons, pions and their related elementary particles pass through our body from the sun every day at a rate of 10,000 muons per minute. Muonsdon't decay they can last forever but they have flavour changes into an electron, a neutrino and antineutrino forcefield making up the Ruach life force, and when they interact with the body's water an electron or positron is produced emitting a light faint ring pattern. The negatively charged particles then also consist of antiprotons, pions and other particles. In 2015 CERN announced that neutrinos could accelerate faster than light. These cannot be seen but their behaviour and that of the electron can be witnessed bending their stream in the natural gravitational fields, through their trail they leave behind as evidence when mobilising this apparatus.

In one example, the apparatus includes up to eight needles circumferentially spaced around a circular housing. However, in use it is not necessary to use all of the needles, and the apparatus can include one or more caps configured to be positioned in the openings to selectively deactivate one or more of the needles. In this regard, the particular needle configuration used will vary depending on the intended application.

For example, when used to treat multiple subjects such as in Wuhan treating four subjects at a time around the apparatus, or to reduce air borne and surface pathogen particles such as in the police drug holding cells, all eight needles can be used. However, when treating a single subject, a four-needle configuration is typically used, with four adjacent needles on one side of the housing nearest the subject being employed to maximize selectivity of the resulting Air Species.

The apparatus is typically configured to generate the pulsed electromagnetic field (PEMF that propels the air species through portals into the air off the needle tips from within the cold air plasma plume and proximate the housing and/or transports the air species away from the housing. Because the human body is now in the centre of the circuit by virtue of the common ground and engineering, the particle-wave duality and air species inside the PEMF and ground arrangement cooperate to accelerate the air species at high speed to the subject, which helps enhance the biological benefits and deep human penetration, resulting in improved biological outcomes. Further because the PEMF has exposure to moisture on the skin it safely reacts and causes pathways into the skin for the transdermal absorption of the species using plasmaporation and electroporation techniques.

This is facilitated by the increased operating voltage, but nevertheless, the apparatus produces less ozone than previous devices, hence making it safe for use in a wide range of systemic therapeutic applications, across all ages and all comorbidities such as treating respiratory conditions, including asthma, chronic obstructive pulmonary disease, emphysema, allergies, auto-immune diseases, sleep apnoea and other sleep problems, pathogen infections including bacterial and viral infections but not limited to only SAR-CoV-2, Ross River fever, lupus, etc., of any mutation, treating cancer including basal cell carcinoma, or breast cancer, or lung cancer or glioblastoma or kidney cancer or liver cancer or treating diabetes, wounds, burns or eczema, dermatitis and psoriasis, blood pressure, hormonal conditions, depression, fatigue to name but a few conditions safely alongside any medications. The apparatus is a physics solution not a chemical solution to systemic conditions so can be used alongside all existing recommended pharmaceuticals, medications and/or vaccines without interaction or a negative consequence.

Specifically, the apparatus is configured to provide a Systemic Selective Adaptogen response to bring about homeostasis and achieve therapeutic and biological benefits across all illness types which are governed by the systems of the body; cause apoptosis; interact across the blood-brain barrier to the Central Nervous System to induce immune, hormonal and all other systemic responses, as an example by interacting with one or more of Serotonin; Melatonin; Lycopene; and Glutathione; plus other mechanisms whilst creating the right environment for further complex immune, apoptosis and protective responses inside cells and systems of the body. For example macrophages and between the cellular and system fluid channels and their biological currents throughout the human body. All these and further reactions require the initiations of the Ruach life force fields to generate photons of light to provide energy to atoms and molecules beyond treatment time up to the following 24-hour period of time and create an optimum environment for the subjects own biological chemistry to respond.

One example is the high energy radicals destroying pathogens using the reactive but safer than ozone and short lived *OH Hydroxyl radical which is known to travel at 1200 eV in 1 nanosecond and can also be formed in the electrochemical oxidation process of H₂O₂. The *OH consisting of one atom of hydrogen and one of Oxygen can only be negative or neutrally charged. When a positron collides due to attraction with the electron, annihilation occurs also creating photons due to the quantum electromagnetic radiation. The photon is the basic unit of light, which travels at the estimated speed of light about 2.998×10⁸ m/s and is created from the junction of the colliding circuits which push the air species and then suddenly pull the electrons resulting in the collision of Ruach life forces inside and/or on the subject.

In one particular example, the apparatus generates a new cocktail of quantum and CAP Air Species that when combined with the PEMF portals and Ruach life force fields provide a retrograde transport along the nerves and this enables them to interact across the blood-brain barrier from the vagus nerves of the respiratory system and impact the brain, autonomic system and the entire central nervous system through both the adaptive and cell-mediated immune responses in particular but not limited to Seratonin, Melatonin, Lycopene and Glutathione impacting T cells and the humoral immune response controlled by the activated B cells and antibodies. These create a systemic selective adaptogen response within the human body.

The ion delivery system of this apparatus, and specifically the connection to the subject, and subsequent internal mechanisms leads to very different modes of operation compared to a traditional ioniser arrangement.

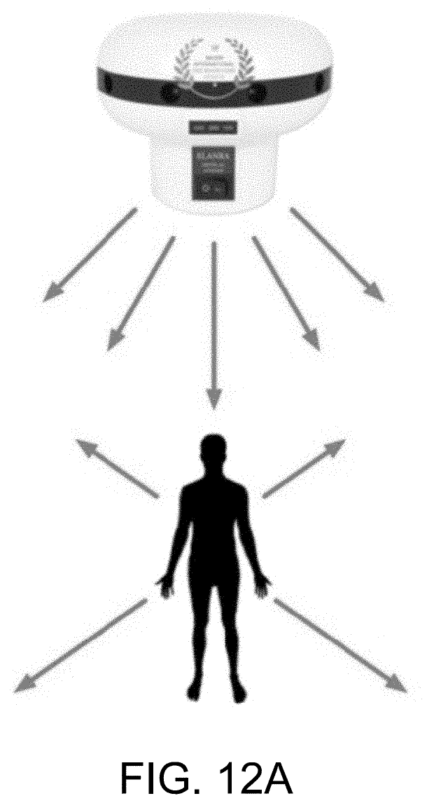

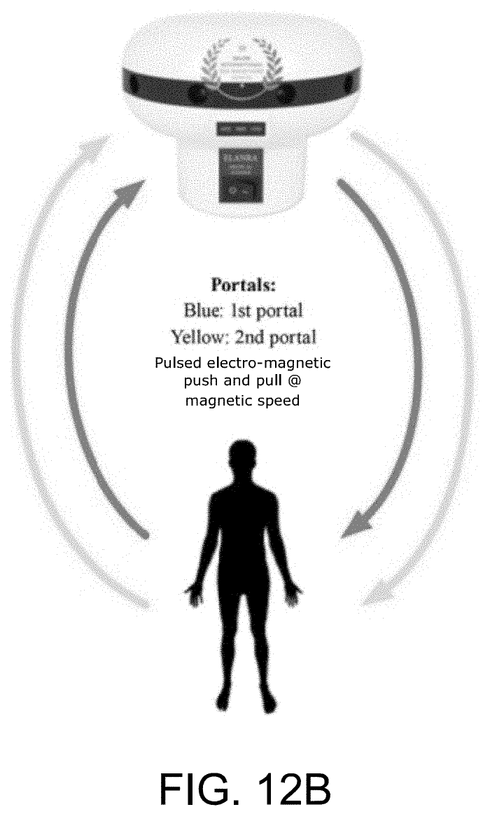

An example of this is shown in FIGS. 12A and 12B, which contrasts traditional ionisers with the current apparatus in relation to their circuit. In the traditional ioniser a negatively charged anode in the ionizer repels negative ions. Insulated and/or ungrounded conductive objects, including the subject, accumulate negative charges in the form of static electricity and begin to repel more negative ions. Electrons and ions search for the closest ground path in the environment, such as metal or ground circuits in the surrounding building, and hence are removed. In contrast, in the current apparatus, a return ground path exists, meaning the subject is grounded and acts as a cathode, attracting the negative ions. Energized molecules within the subject are ionised, with the molecules remaining in the body, whilst the electrons are ripped away suddenly returning back to the apparatus via the ground circuit which are referred to as portals. This represents the inner loop (or first portal) in FIG. 12B. The returning electrons are accelerated to form a secondary electron circuit represented by the outer loop (or second portal) in FIG. 12B. In this instance, the second portal leads to electrolysis arising in the cathode (the subject), where H₂O can be split into H₂ and O₂ gases, as will be described in more detail below. Thus, in the current arrangement, O₂ (and other air species) are ionized at the anode and accelerated towards the subject. Meanwhile, secondary electrons being more energetic then give rise to the generation of hydrogen H₂ within the subject, leading to multiple modalities being the whole body's master metabolizer.

Thus, in the current apparatus, the needles produce electrons and charged ions within the corona plasma plume at high potential, but they won't go anywhere without a return path, they will simply scatter into the environment. The charged ions leave the needle, travel through the air as a particle-wave duality with the carrier frequency and other quantum waves and are driven by the PEMF and other forces towards the lowest potential they can find. This is anything connected to the electrical mains system, including the actual earth or in this case the custom designed grounding ring within the apparatus. When the ions collide with a ground connected conductive surface (the subject), they lose their spare electron and it causes current to flow through the ground path. The circuit is completed because of the engineering, which draws the electrons from the needle through the common grounded human and back to the apparatus grounding ring via a novel isolation bridge which pulls the electrons into that grounding ring like a very powerful magnet, creating the first portal. The now more excited electrons find their way off the grounding ring and back onto the needle assembly and are then raised to high potential and ejected again into the air through the plasma creating the ‘secondary electron’, or second portal. The electron has returned to the start of the circuit again, and now creates the secondary life of the portal which becomes a repetitive cyclical process but at very high magnetic speeds. The secondary electron appears to be carrying a higher momentum by the time it enters the 2nd portal, because it has travelled through its first portal process in which it is believed to have gained substantial energy transfer.

For the desired therapeutic health benefits, the ions must meet the human subject at extremely high speeds to retain their purpose, that of being an extremely small (less than atomic) size, speed and agility and hence the mechanism allows the crossing of the blood brain barrier. It is known in physics when a surface (human subject in this case) is hit with charged particles of high kinetic energy that emission of those electrons occurs and it's recognised as secondary electrons, further the solid matter could release a greater number of electrons, as described for example in Dekker A J., “Secondary Electron Emission”, Solid State Physics, Springer Link 1981, p.418-45. In this instance, quantum tunnelling can allow penetration of the subject both ways so this theory can explain part of the secondary electron behavior. If the human is properly grounded, they will receive more of the ion current passing through their bodies. For this to happen in practical terms, they may sleep on an electric blanket, a grounding mat, have bare feet in their office or wear a wrist strap connecting the body to ground, so that the PEMF will drive the ions towards their body. This force can be perceived as a gentle quiet “ion wind” (breeze) flowing from the needles.

Conversely, if the subject is not grounded, a lesser amount of ions will flow towards them and they will feel much less of the “ion wind” if at all. In this instance, the ions will tend to accumulate to the body as a static charge, with the result, that if the human does subsequently touch a surface they will feel a non harmful zap when they touch a conductive path to ground; similar to when touching a car door on a dry winters day.

The charge is designed to return back to the apparatus through the subject as long as the subject and apparatus are properly connected to each other in unison, which doesn't need to be via a cable, but instead is via the ground path as presented. The secondary electrons move at magnetic speed from the human back through the portal to the CCAP device without the need for using more AC power and consequently having a possible adverse event of excess ozone production which could be toxic for human health. Experience and observations of the electron trail suggest the molecules don't go any further than the subject. Because the ion is a molecule with a spare electron, once that molecule reacts with the body's chemistry in some way, the ion no longer exists as such, because the electron then escapes but it does so as a conductive charge, whilst the molecule has been absorbed by the body.

The electron escape is immediate and ripped away from the molecule with sufficiently sudden force to find its ground back through the portal and to its grounding ring, which it finds in the specially made place within the device. That completes one circuit, but it is a continuous cycle. This process occurs because the conductive charge of the electron leaves a trail that the engineers can visibly see, whilst the human body responds with a plethora of therapeutic positive benefits from its energetic retained molecules. Some kind of biological fusion is taking place at the ion when the electron is ripped away from the molecule suddenly which explains the creation of photons and the particle-wave duality. In the field of quantum mechanics, they discovered that certain sources of photon purity can influence its particles and waves, and that a particle can become a wave and a wave can become a particle in right conditions, as described for example in Yoon TH, Cho, M. Quantitative complementarity of wave-particle duality. Science Advances. 2021;7. This extends the originally proposed theory of wave-particle duality concept Wootters W K, Zurek W H, “Complementarity in the double-slit experiment: Quantum nonseparability and a quantitative statement of Bohr's principle”, Physical Review D, 1979;19(2):473-84. This process explains the quantum tunnelling effect that would take place for the electron to move through the body from its ion and back into the portal for ground.

As mentioned, in one example, this leads to the ability to generate hydrogen within the body through water electrolysis.

In this regard, hydrogen has a key role in the human body's metabolism and homeostasis. Hydrogen is known in tablet or water form to benefit the human body and recently is considered in a molecular form for therapeutic use for treating COVID-19. It is the master stabilizer of the body's water and the unique element, which closes the twists of the DNA chains. The body stores up pools of H₂ supplies for the body's future use and H₂ protects the body from free radical damage. It is a powerful antioxidant and makes up about 10% of the body's mass Alwazeer D, Liu F F, Wu X Y, LeBaron T W, “Combating Oxidative Stress and Inflammation in COVID-19 by Molecular Hydrogen Therapy: Mechanisms and Perspectives”, Oxid Med Cell Longev, 2021; 2021:5513868.

Subject results strongly suggest there is a master metabolic mechanism in action, which is needed to explain their positive long-term results across a large spectrum of human health. The novel circuit with the theory and mechanisms behind water electrolysis (of which the body is mostly made up of water), quantum tunnelling, Maxwell's equations and an extensive amount of multidisciplinary sciences with results in human health are an encouraging explanation that this medical device is creating hydrogen (H₂) by splitting some water molecules inside the biology of the human.

Water, being very stable, does not naturally decompose into oxygen and hydrogen. Only once an energy is used to decompose water is it possible to move to its component elements. There is currently no known way for scientists to experimentally test this as yet, but the theory and subject results lead to a reasonably high probability that this technology creating the right environment for hydrogen (H₂) production and therefore H₂ inside the human body.

Testing of the electron trail suggests it is likely that this occurs at the junction of the centre circuit between the two portals, where the ion and molecule reacts inside and with the body's chemistry. Thus, in the current arrangement, the charged molecule stays in the humanas evidenced by the myriad of positive therapeutic results, and engineers have demonstrated evidence of the electron being removed out of the body, specifically returning to the device via a return current path. This is evidenced through degradation of the anode within the cold air plasma generating apparatus in specific areas related to the electrons return path from its travel. It is also possible to see a trail of tunnelling back to the needle assembly as a secondary electron by creating deliberate hair line cracks for its overflow to leave its trail as it travels.

The secondary electron consists of a higher charge and speed. Although it cannot be seen or measured, it can be tracked via its pathway of degradation to internal components of the apparatus. The combination of the events from the secondary electron and the circuit with this increased density create the right environment for Hydrogen (H₂) to be initiated via water electrolysis inside the human biology. In one example, the internal environment of the apparatus is configured to assist the electron in reaching the grounding ring and subsequently returning to the needle assembly to become the secondary electron, whilst protecting the balance of the high voltage components from its subsequent increased ability to cause degradation.

Internal components have shown to be compromised with the application of conformal coating due to the inability of such current modern methods to deal with electrons and other atomic size and speed species to avoid the smallest of hairline air bubbles around the components when applied, and hence cannot be used. With the current arrangement and the production of cold air plasma at the needle tips and the subsequent improvement in higher ion density, flow rate and magnetic pull with fusion that strips electrons from air molecules to generate hydrogen (H₂), and attraction of the electrons from body to ground, a new solution is required.

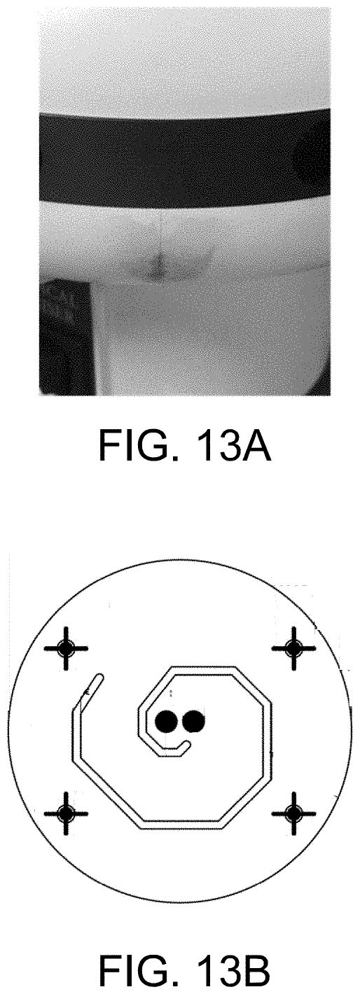

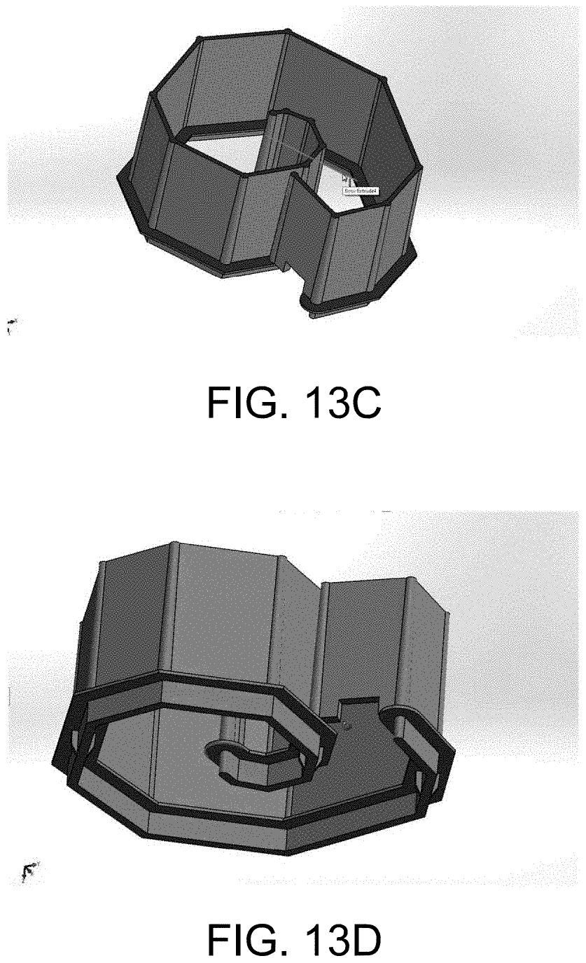

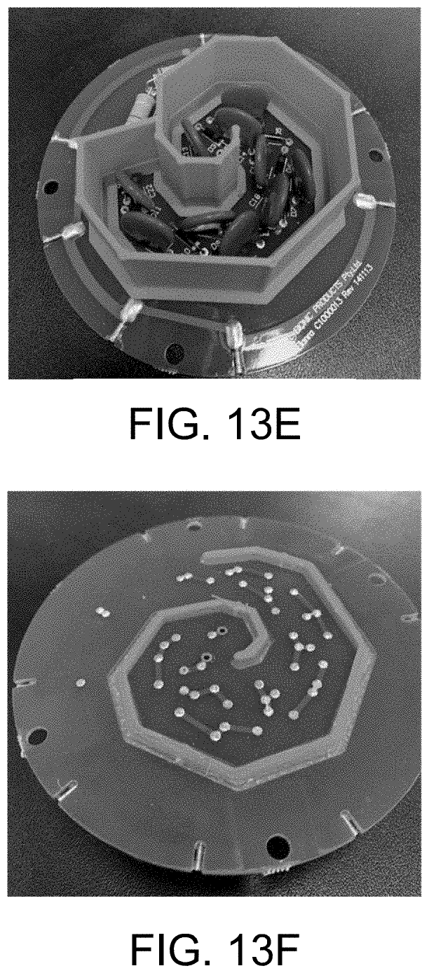

To demonstrate this, various deliberately damaged cracks were created into an exterior of various devices until an even width vertical hairline crack was witnessed directly outside and over the grounding ring and between two needle assemblies, as shown for example in FIG. 13A. The housing degradation around the crack on the outside displays naturally formed shadows that show the electrons behavior on the ‘inside’ of the apparatus as its overflow ‘leaks’ through the evenly distributed crack. On the lower half of the crack a concentrated darker shadow is seen which is directly adjacent to the internal grounding ring, this evidences the return of the electron from the body onto the grounding ring inside the device at high concentration and speed. On the top half of the crack a gentle shadow shows electron spread defraying towards the left upper quadrant and towards the right upper quadrant. These gentle shadows evidence the electron journey after escaping the grounding ring and being attracted to each of the needle assembly's which are above and away from the grounding ring and further in from the housing. One group of electrons fan towards the left needle assembly and the other group of electrons fan towards the right needle assemble. This sweeping movement to each side is evidenced by the electron overflow shadows through the crack where the electrons still move in the direction of their atomic intended journey; when they reach each needle assembly, they are then charged up to high potential again and become the secondary electrons of higher speed. The smokey appearance to the housing, is caused by the electron overflow passing through the cracks travelling from the inside to the outside where it leaves us its trail of how it is behaving on the inside. The upper shadows being lighter in color is expected because to escape the grounding ring which touches the housing they need to travel further inwards of the apparatus to the needle assembly so it is expected to see a smaller portion of overflow available to seep outside the crack. The seeping electrons leave a trail behind of their presence, their density and the direction they are heading. Accordingly, degradation of the components and housing can be used to track the electron species behavior on the inside and monitor its consistency, which was confirmed.

Accordingly, an insulator is provided within the housing in order to isolate the high voltage components, thereby reducing the impact of these more powerful electrons on the housing and assist in directing electrons to travel to the needle assembly by removing alternative pathways. This in turn enhances secondary electron creation. The insulating member typically has a convoluted shape enabling this to surround the high voltage electronics while providing a current flow path to the needles.

In one example, during testing, an insulator was provided in the housing in the form of a non-flammable approx. 23.5 cm long trimmed and 25 mm high CD2516 cable electrical conduit ducting, which was manually attempted for testing, to wind around the HV board components for protection and tested again. It was found to reduce the degradation and did not interfere with output measurements, but requires a high degree of labor to implement into each device.

An insulating member was therefore created using the same external grade plastic used for device housing. The insulating member has a convoluted shape, shown in FIGS. 13B to 13F, so that the insulator surrounds the high voltage components, thereby reducing electron flow through the housing. However, this still provides a current flow path to the needles, allowing the electrons to find their way to the needle assembly, after finding the grounding ring from its removal from the charged molecules inside the human and then becoming the required secondary electron at the needle tips to create the higher required charge rate and speed needed for water electrolysis to occur inside the biology and created Hydrogen (H₂) using the body's own water for therapeutic benefits H₂ being the master metabolizer for all systems of the human body to achieve homeostasis.

Maxwell's theory shows that the atomic magnetic system functions at right angles to the molecular electrical system and that each of them changes with time at a rate which is proportional to the curl of the other. However, here the field is altered so that it points in a second direction because the portal pulls the electron back towards the apparatus off the ion in the body. Therefore, its curl is now perpendicular to the two directions and so its rate of change in time is going to be in the same direction as itself. A cold fusion is shown to be taking place inside the human at this separation of the electron, which explains the creation of the life force light photons referred to as Ruach life force inside the human, which underpins the repeatable and consistent results seen across many targeted and untargeted health conditions. This leads to a reasonable argument that this same Ruach life force and produced muons and pions is also responsible for the forces which hold our ‘plank atoms and nucleons’ together.

Given the human body is mostly water, it is feasible that water electrolysis occurs given the right environment. These reactions require a biological cell potential to be reached of +1.23V or more for electrolysis to occur, as described for example in Carmo M, Fritz D L, Mergel J, Stolten D., “A comprehensive review on PEM water electrolysis”, International Journal of Hydrogen Energy, 2013; 38(12): 4901-34, as well as Shiva Kumar S, Himabindu V., “Hydrogen production by PEM water electrolysis—A review”, Materials Science for Energy Technologies, 2019; 2(3):442-54, or Acar C, Dincer I., “Hydrogen Production”, Comprehensive Energy Systems, 3: Elsevier Inc; 2018. p. 1-40. Even though this is thermodynamically favored in water electrolysis, it's still not fast enough to break the H₂O molecule unless it is ignited by some kind of ‘spark’. The ripping of the electron off the ion can cause a cold fusion spark at the biological level and the electron reverses its direction leaving the spinning atomically hot molecule, and quantum tunnels back out of the body in the direction it came. If the cell potential is achieved, then the new energy or Ruach life force created at the molecule and the photon formed from that will be a spontaneous reaction, because the body is very suitable for water electrolysis to occur. First of all, because the body's systemic biology is made up of significant amounts of water, buffer system-managed salts and buffer system-managed pH levels, and secondly because of the additional pulling power from the apparatus portal actions ripping the electron out of the body. So, the body then becomes its own closed circuit facilitating the current flow over and over and consequently being an ideal environment for water electrolysis and the initiation of a cold fusion spark at the biological level. Further, because the body's salty water is engaged by the current, engineering theory shows that the body will become a closed circuit in its own right, and interestingly it is then a closed circuit within a larger repeatable closed circuit at close to the speed of light (magnetic circuit speed) over and over until turned off.

There is another mechanism that also explains the metabolic sustained energy from long term (>1 year) positive subject results, it shows that the probability of this cold fusion taking place is highly plausible. It was reported that non-linear coupling between dipole oscillators can channel energy into a single coherent oscillator as described in Chen C, Zanette D H, Czaplewski D A, Shaw S, Lopez D, “Direct observation of coherent energy transfer in nonlinear micromechanical oscillators”, Nat Commun. 2017; 8:15523. Occurring inside the body, then the fluids, interstitial fluids, water channels and the currents of bodily fluids within the body can also resonate, and this has now been shown to activate cells, as described in Kylychbekov S, Song H S, Kwon K B, Ra 0, Yoon E S, Chung M, et al., “Reconstruction of plasma density profiles by measuring spectra of radiation emitted from oscillating plasma dipoles”, Plasma Sources Science and Technology, 2020; 29(2) or Tyshetskiy Y, Vladimirov SV., “Quantum-tunneling-enhanced charging of nanoparticles in plasmas”, Phys Rev E Stat Nonlin Soft Matter Phys, 2011; 83(4 Pt 2):046406.

The body is capable of then storing up that energy for later use, and activating the energy at biological temperature, which then enables the body to control its own chemical reactions as it needs and when it needs, as described in Trixler F., “Quantum tunnelling to the origin and evolution of life”, Current organic chemistry, 2013; 17(16):1758-70, Montagnier L, Aissa J, Giudice E D, Lavallee C, Tedeschi A, Vitiello G., “DNA waves and water”, Journal of Physics: Conference Series. 2011; 306 or Montagnier L, Del Giudice E, Aissa J, Lavallee C, Motschwiller S, Capolupo A, et al., “Transduction of DNA information through water and electromagnetic waves”, Electromagn Biol Med. 2015; 34(2):106-12.

The energy can potentially be stored surrounded by or within water molecules in a similar way to how a piece of DNA was diluted in pure water at a weak EMF of 7Hz, and then its memorized resonance was recorded on a USB and rebuilt itself elsewhere in another vial of water in another geographical location as described in Montagnier L, Del Giudice E, Aissa J, Lavallee C, Motschwiller S, Capolupo A, et al., “Transduction of DNA information through water and electromagnetic waves”, Electromagn Biol Med. 2015; 34(2):106-12. Notably this is the electromagnetic spectrum level that this apparatus resonates and its particle-wave duality PEMF and vibrations specialize in; thereby showing outside this apparatus in human results that communication and memory takes place at this extremely low end of the resonating spectrum for the human biology (<10 Hz in this case) and it requires PEMF and water. Further electron tunnelling has been found to have a short transversal time and in water allows the H₂O dissociation mechanism to take place.

Water radiolysis, particularly involving ionised species, exhibited improved hydrogen-deuterium kinetic isotope effects and splitting of the H₂O molecule at room temperature, which was attributed to such a quantum tunnelling phenomenon, as described in Nikitenko S I, Di Pasquale T, Chave T, Pflieger R., “Hypothesis about electron quantum tunneling during sonochemical splitting of water molecule”, Ultrason Sonochem, 2020; 60:104789. In the field of quantum electrodynamics it suggests in certain situations similar to this apparatus environment that the lower energy coherent state of the electron clouds of water molecules (present in the biology from the hot spinning molecule) oscillate between its excited configuration and ground configuration so causes one electron per molecule to be free (binding energy of 0.4 eV), this is because the molecule coherent oscillation is in tune with the PEMF, see for example, Giudice EDea, “Coherent structures in liquid water close to hydrophilic surfaces”, Journal of Physics: Conference Series. 2013; 442(012028).

The mechanisms discussed are further supported in chemistry, as described in Scott K, “Chapter 1: Introduction to Electrolysis, Electrolyzers and Hydrogen Production Electrochemical Methods for Hydrogen Production”, Energy and Environment Series 2019. p. 1-27, at the polar ends of the portals, which as shown in FIG. 12B, the device acts as an anode and the human being as the cathode in this circuit. Reduction takes place at the cathode (human) which splits the water molecule and creates hydrogen (H₂) gases, but oxidation takes place at the anode which is the apparatus. The electron racing to the anode is received by this electron acceptor and the charged electron leaves behind a trail of its movements, which can be viewed by observing degradation at the anode destination created for it. This substantiates the physical evidence of the return path of the electrons and therefore provides for the understanding that the portal configuration contributes greatly to the required spark from the Cold fusion which initiates the cell potential required to split water and create hydrogen (H₂) production inside the biology whilst the electron is quantum tunnelling back to its ground.

Water electrolysis shows that at the cathode H+ + e− becomes H, but that it is unstable, so it combines with another. So H+H then becomes H₂, and twice the amount of hydrogen in the body's water will be formed to the amount of oxygen which is formed back at the device.

A number of clinical studies have been performed and are underway using the above-described apparatus with the new utility of use and examples of these results and some variables will now be described. Further the apparatus is currently used by 72 subjects as at August 2021 across 54 illnesses in decentralized hybrid clinical case study trials where each study is individualized and subjects record their observations from 30 days up to 12 months. 100% of the subjects suffer more than three comorbidities showing the systemic effects of illnesses in our modern times.

### Asthma Case Study (2,288,993)

A 60 year old female patient diagnosed with Asthma in 2010. The patient's conditions reported are asthma and scoliosis which decreases the (L) lung capacity by 25%.

The patient agreed to record baseline asthma conditions before using the device over a period of 2 weeks and it was determined the main triggers were environmental: dust, exhaust fumes, sudden change in temperature, wind and is sensitive to minor environmental changes. Patient manages condition using 2 puffs morning and night of Seretide 250 mg and Ventolin when needed. When these are not enough, patient uses the nebulizer every 4 hours until symptoms have subsided.

The patient was treated using this apparatus positioned 1.5 mtr from the centre of the patients pillow each night as she sleeps. Patient's age and normal stature would normally mean we use an immune type of setting. However, patient has communicated being “oversensitivity” to changes in air conditions and so weaker settings were trialed to be cautious, using settings of 4, 6, 7, 8.

Following treatment, the patient has reported that the usual cough she had during the day has mostly disappeared. She has stopped taking her steroids Seretide, and only has Ventolin very occasionally.

### Asthma Case Study (2,289,000)

In another study, a 40 yr old male with mild asthma for 30 years was treated. The patient is 89 klgs. Non-smoker. Exercises regularly. Room temperature & humidity average 22 degrees and 50%.

The patient has had mild asthma for 30 years. Takes Salbutamol reliever when required and Clenil Modulite 200 preventer inhalers (2 puffs per day). Allergens that make the patient worse include Cat, Carpet, Cold air.

Results: Comparing a 14-day base line without using the device; to a short term of 8 days with the device the patient's O2 levels improved an average of 0.4 points with the device across evening and morning measurements (using pulse oximetry). Peak expiratory flow improved by an average of 17.5% in the evening and 11.4% in the morning with the device on (using a peak flow meter). Shortness of breath improved by 48% in the evening and 20% in the morning (self-assessed rating).

Fatigue levels deteriorated in the morning by 23% (self-assessed rating), which is an expected result for short term analysis. It tells us the patient is responding and sleeping deeper at times during the night, with the impact creating more perceived tiredness first thing in the morning,

### Basal Cell Carcinoma Study (2,288,991)

A patient with a Basal Cell Carcinoma on the back of the right ear and cartledge was treated using this apparatus.

The patient had previously chosen not to have any medical treatment, so after about 6 years the carcinoma had become a large ulcer type wound weeping behind the ear and the front was inflamed and swollen. The wound was open and painful which bled every few hours. The patient had previously been advised there was no other treatment than to have the whole outer-ear removed surgically, which he refused.

This apparatus was placed within 30 cms of the patient's ear on the bedside table next to the bed on a high setting for 2 hours per day during the day (an intense treatment at 3-4 Hz) and 1 meter from patient's head during the night while the patient was sleeping on a lesser setting (a respiratory systemic treatment mild strength at 4 Hz). During this process, the wound was exposed to the species intensely during the day and the body's systemic treatment is exposed during the night as he sleeps.

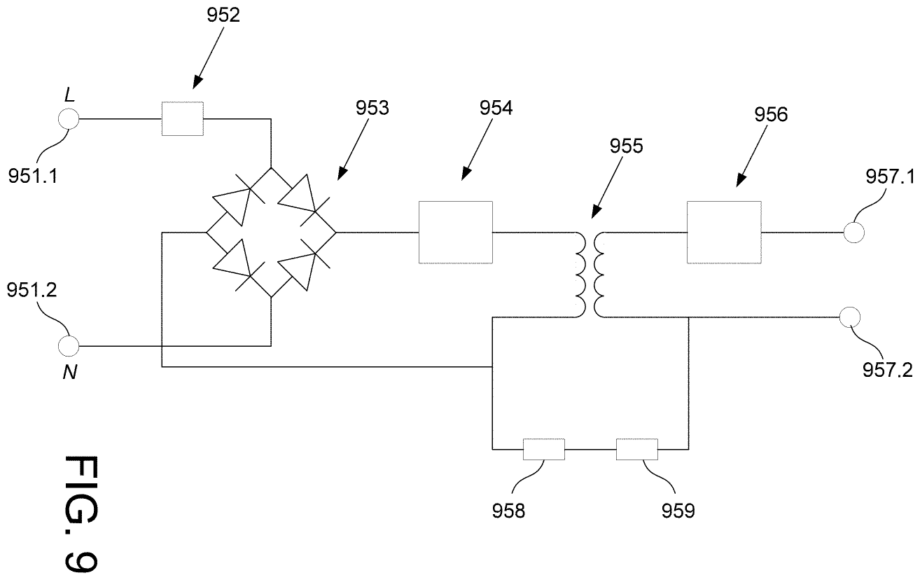

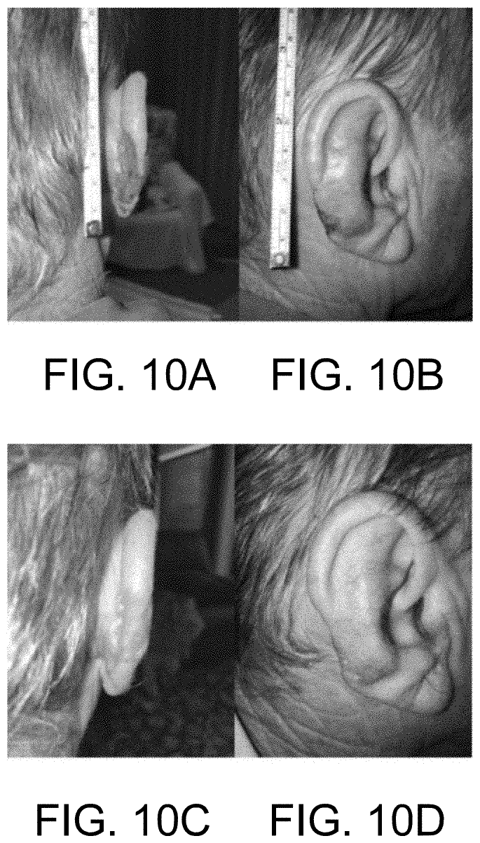

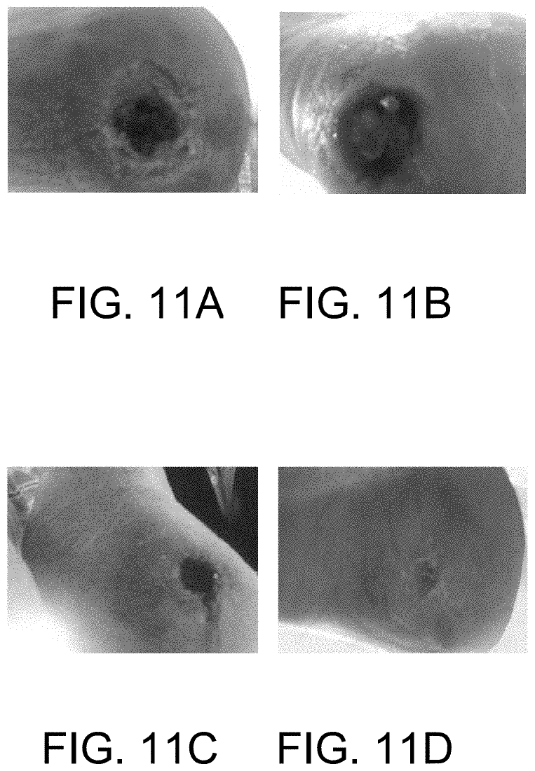

Within 10 days the wound was shrinking in size and no longer weeping. There was no pain anymore. Within 20 days the wound stopped all bleeds. After 30 days the wound is almost healed over. The patient has reported that this is the first time in 5 years that there is almost no wound behind his ear. Comparative images pre and post treatment are shown in FIGS. 10A and 10B, and 10C and 10D, respectively.

As the wound has improved well, treatment is now focused on treating the cartilage area by changing the way the 2-hour intense treatment of the patient is conducted. Instead of the device being placed 30 cm ‘behind’ the ear, it was now to be placed directly ‘facing’ the ear and side of face. The patient does one hour with the device positioned behind the ear and then one hour with the device directly facing the ear at the side of the face and to wet the ear surface with water or moisturizer. The patient wore full eye protection for caution.

During the treatment, at the 9-week mark, the patient was requested to use a conductive grounding mat (metal mesh) under the head while doing the 2-hour intense treatment as per previous instructions. The reason for this was to encourage a more focused treatment by improving conductive grounding. Amputation of the ear is no longer required.

### Blood Phagocytes Research (UZZA 00015)

A study was performed to investigate the ability of this apparatus to boost the immune system through its ability to assist Phagocytes in the blood become excited and respond more effectively

A test was done in vitro using live blood from a patient suffering chronic fatigue. The live blood sample control was spread onto a microscope glass slide and viewed live immediately to show pictures of Phagocytes which were lazy and not moving in the blood, rather just floating amongst the cells.

The same blood sample was then placed in front of the above-described apparatus for a period of <30 seconds and again observed under magnification, which demonstrated the Phagocytes were now excited, illuminated more brightly and shining like a diamond, as we watched one phagocyte begin to open up and eat debris from within the blood environment, acting in a much more energetic way, as they are intended too. This was an effective sample experiment which simply showed the Phagocytes being more responsive in this particular raw experiment.

### Glioblastoma Brain Cancer (2,289,005)

A 58 year old male of 105 klgs was diagnosed with glioblastoma. Some tumours were operated on and the remaining ones could not be operated on. The patient was sent home for palliative care with less than 8 weeks to live. The patient tried the above apparatus with the intention it would assist his breathing and snoring at night. Settings used for the patient were a strong output but a 2 Hz low frequency.

Patient reported improved sleep and more energy. After 5 weeks of treatment his scans showed the glioblastoma had stopped growing. By week 7 his doctor informed him the tumor was shrinking. At week 12 his doctor believes he is in remission, he has gone back to playing his favorite sports and living his life. Patient made no other changes in his life nor was he taking any medications.

### Brain & Lung Metastasized Cancer (2,288,996)

A 25 year old male patient with terminal metastasized brain and lung cancer was treated using this apparatus. The patient had had numerous surgeries and the cancer had metastasized to the lungs, meaning he only had half a lung left, following surgery. His Oxygen levels were 88, and surgeons could not improve its level, he was sent home for palliative care for the 7 expected days he had left. Investigations were requested by the surgeon and performed to see if this apparatus could raise his blood O2 levels in the interim.

An oximeter was used to start testing the patient's blood oxygen levels during treatment with the apparatus. When the apparatus was first set up, the patient's O2 reading hovered between 86-88, he was pale and weak, but in good spirits. The apparatus was placed (by the patient) at about 20-30 cm from the patient's breathing space as he sat comfortably whilst breathing, still no change. After 36 hours of treatment the patient's blood Oxygen levels had eventually increased up to 95. This blood oxygen level has been maintained at that level since, and he is still with us 14 months later and in remission.

### Breast Cancer (2,288,998)

A 52 year old female peri-menopausal patient with a clear mammogram, and no palpable lump or symptoms was diagnosed with a stage II Infiltrating Lobular Carcinoma Breast Cancer 1.2×0.8×0.9 cm at a depth of 5 cm inside left breast at the 3 o'clock position following routine ultrasound.

The patient was treated using the device daily with two hourly intense treatments during the day at 2-4 Hz and high output 20 cm from the left breast tumor site area as a transdermal treatment, and 7.83 Hz medium output for respiratory application at night-time at a distance of 1-1.5 meters. She maintained that daily treatment through to the date of surgical removal 4 weeks later, monitoring tumor size changes with multiple ultrasounds in the interim.

Results demonstrated throughout the almost 4 weeks that the tumor was reducing in size from 12 mm to 9 mm from three diagnostic ultrasounds, a mammogram, ×3 core biopsies (12 mm to 21 mm) and CT Scan measurements using same machine and radiographer, before eventual surgical removal and subsequent final measurement and pathology results. The tumor reduced by about 33.3% inside the period of testing to show efficacy during this novel treatment and had further altered the tumor type from an infiltrating ‘lobular’ carcinoma to a less severe ‘ductal’ carcinoma.

Upon surgical removal at the four weeks mark, the pathology report showed the tumor was now absent of cancer cells from all tested tissues. ‘Absent Ductal carcinoma in situ’ and ‘Absent lobular carcinoma in situ’. The lymph nodes were also negative of any cancerous cells. And all margins excised were also negative for any cancerous cells.

It should be noted that the patient was ER+, PR+ and HER− and considered low risk and chose this device for her treatment plan. The patient undertook the treatment regime to see if the device would make a difference to her tumour prior to her premeditated decision to still continue with the surgical removal as recommended.

The patient declined standard of care' radiation treatment offered after she discovered that the side effects are ‘permanent heart and lung damage’, ‘chronic fatigue’, ‘burns to her chest and back’ for the rest of her life. If she proceeded with radiation her>95% 5-year survival rate would increase in her case by only 2%, she thought the side effects would be worse than the slight improvement in survival. She also declined hormonal treatment due to side effects.

The patient continues to use the device at night-time and continues with intermittent focused treatment (3 days per week) to both breasts at the above intense treatment for two hours at a time. She has regular ultrasounds of both breasts and is living a normal life.

### Breast Cancer Wound Study (2,288,995)

A 52 year old female patient diagnosed with Stage II Lobular carcinoma breast cancer had surgery to remove a breast cancer tumor from her left side of breast nipple region. During surgery the same surgeon also removed x 3 sentinel lymph nodes following radioactive tracer from the left side of the breast near the armpit for precautionary analysis. The incision of the breast was deeper than the incision of the armpit area, but both were about the same length performed by the same surgeon on the same day, during the same operation, in the same room and the same environment. The patient left the armpit cut above the lymph nodes bandaged for the full 4 weeks, blocking its exposure to the air species.

The patient was totally bed bound for one week for recovery and mostly bed bound for the following 3 weeks. The patient removed the bandages from the deeper breast wound and deliberately exposed it to air species with energy fields using the above-described arrangement within two days of the surgery. The apparatus was used for 22 hours per day at the bedside a ta distance of 1 meter on 4 Hz and a medium strength, and about 2 hours in the middle of each day on the ‘Intense use’ 3-4 Hz with a stronger setting and a closer distance of about 20 cm.

After four weeks, there was a substantial reduction in both scarring and color of the deeper breast surgical wound that was exposed to the air species and energy fields, compared to the wound that was totally covered over with a bandage from the same operation.

### Chronic Obstructive Pulmonary Disease (COPD) & Sleep Apnea (2,288,992)

A COPD and sleep apnea study was performed to compare the oxygen content in a subject treated with a commercial CPAP ventilator or the above described apparatus. The study ran for 5 days, with a total usage time of the above apparatus being 20.5 hours, versus a usage time of the commercial CPAP ventilator of 23 hours.

The commercial CPAP ventilator resulted in a 12.72% reduction in the average oxygen content in the patient's blood, whereas the above apparatus resulted in a 34.27% increase in the average oxygen content in the patient's blood over the same time period. The patient stated they felt better in the morning using this apparatus. This study demands further investigation for a larger group of COPD and Sleep Apnea patients.

### COVID-19 (2,289,015)

These apparatuses were provided in Wuhan Hubei Province and Hebei Province PRCon ‘compassionate grounds’ all patients are of Chinese origin during January to February 2020. At that time reports were that on average about 25% of the now named SARS-CoV-2 infected patients passed away. Hospitals were crowded quite rapidly. Cross infection was high as the doctors were working out the transmission of the perceived SARS type virus, within a short time frame the PRC provided medical team support from other provinces, at which time the situation was managed and quickly controlled.

The above apparatus was used in both a 4 needle and an 8 needle arrangement, with a patient in bed on either side of the device or around the device without wearing a mask in a mostly closed room and receiving the same species in the room where they lay. Settings were all different in accordance with patient O₂ levels and variables assessed for each. Estimated patients about 200. Two intubated patients were treated using a transdermal approach through the centre back towards the lower half of the lungs at a 20-30 cm distance. 100% of the patients who used the device did not deteriorate, all were discharged. Further our chairman on site only had a paper mask and swimming goggles as PPE and using the device nightly, did not get infected. Patients were also treated by doctors with traditional Chinese and western medicines.

### Diabetic Foot Study (2,288,994)

A patient with severe Type 2 Diabetes gained a necrotic heel, which is a bed sore or pressure ulcer/sore that is common for late-stage Type 2 diabetics. In this case the wound was so deep, one could view the heel bone through the weeping hole through the skin.

The patient was a resident at a nursing home that used conventional methods of treatment: creams, acid baths, bandages, regular dressings and wrapping with scalpel cleaning. The patient was prepared for the possibility of amputation if the wound couldn't be rectified due to its severity. The patient also had a stent inserted into the main artery of the leg to unblock and allow improved blood flow into the foot. The stent helped, but not enough to heal this wound which was getting worse after 12 months of traditional treatments. Even with increased regularity of acid cleans and re-bandaging the patient's condition was worsening. He refused amputation and demanded use of this apparatus.

The above apparatus was placed within 30 cms of the patient on a wooden stool at the end of the bed. It was set on a high output with a setting of 32 Hz in the first month and then 4-8 Hz thereafter. Prior to using the medical device, the patient was not walking and bed bound except for toileting and showering. His meals, room temperature and environmental variables are stable. The patient remained in bed until the wound was completely healed, during the process the wound was exposed to air species and energy fields using the transdermal approach. The apparatus was most effective at close proximity of 30 cm from the affected area. The leg was also slightly elevated with use of a pillow and waterproof mat placed underneath to collect any discharge from the wound as it healed. The leg was covered by an A frame and a blanket draped over it to prevent visitor or air conditioner bacterial air nuclei accessing the foot wound.

Within 3 weeks the depth of the wound closed over the bone making it less visible, formed scabs around its edges and ceased to weep excessively. Within 4 weeks the width of the wound decreased in size from 10 cm to 3 cm and weeped slightly. The wound was never touched with any material other than scalpel of rough scabs around the edges to expose the raw skin to the air species and energy fields. Within a total of 11-12 weeks the wound was healed to allow the patient to have full use of the leg and foot. Amputation was no longer required.

### Eczema Case Study (2,289,001)

A 72 year old female patient had eczema on both ankles and inside legs for the first time, it was painful and itchy, resulting in the patient needing to bathe in the middle of the night to cope with the pain. Her doctor put her on prednisone, but she couldn't tolerate it.

The patient used the above apparatus for a daily intense treatment for two hours with the device positioned 30 cms from her eczema using a strong setting with 8.5-10 Hz and then to change the setting and have the device next to her bed for her normal night-time use which in her case was a medium strength and 11 Hz at 1.5 meters.

After 2 days of treatment the patient reported the eczema was feeling less rough and she felt like something was happening. After day 8, the patient reported the eczema as feeling dry and the itch had been further reduced. By day 14, the patient reported that since day 12, she didn't have to bathe her legs at night anymore and only had to put moisturizer on one leg during the night. By day 39, the color of her legs is back to normal, pain is gone and itch has gone, she is living her normal life.

### Emphysema Case Study (2,289,006)

A 38 yr old male patient of Chinese origin with diagnosed right lung lower lobe emphysema is being treated. The patient recently quit smoking. Using Oximeter to measure before and after blood O2 and a peak flow meter.

Patient conducted a one-month baseline measurement and monitor results over a 12-month period. Blood Oxygen levels are an average 98, and Peak flow is an average 584 which are both good and stable at this point in time. Following measurements are from patient self-graded improvement or deterioration per day, using a 6-point numbering system and comparing his PM results to his AM results. Changes by the morning and ongoing:
Fatigue improved by 54%
Shortness of breath improved by 24%
Coughing improved by 50%
The amount of mucous produced improved by decreasing at 77%

### Covid-19 Anosmia—Case Study (2,289,018)

A COVID-19 40 yr old female patient of 65 klgs from Romania was treated. She has used the earlier device technology but was not responding. New apparatus was sent to her when she informed us of her diagnosis of COVID-19. Apparatus is being used for 10 hours/day at night-time.

Medical diagnosis: COVID19 Positive. She was sick with low O2, high temperatures, soft bowels, very painful muscles in her legs and then after her temperature stabilized she lost her sense of smell. Doctor told her to use symptomatic treatment and follow health state advice and ring emergency if she deteriorated. Doctor prescribed Movalis for her severe leg pain but she could not tolerate it. Paracetamol to keep her temperature down (only took 3 pills). Before she lost her sense of smell, she said she began to perceive strange smells for no reason. One week after her COVID symptoms decreased she had an unusual lingering cough for 3 weeks.

Other medical conditions: Has Hypertrophic allergic rhinitis. Small kidney stones. Heavy metal poisoning (palladium 2.5 more than normal) right knee pain and food intolerances.

The patient was treated using the above apparatus at 1 m distance from herself for 10 hours overnight each day. During the day the device remained on for the further 14 hours, however, she moved around her apartment in quarantine and was outside the two metre biological range, remaining within 5 meters of the device.

Fast recovery was seen from COVID19 symptoms within 3 days of using the apparatus. Anosmia symptoms then started the day her main COVID19 symptoms ceased and deteriorated further over 3 days with complete loss of smell experienced by day 4 after COVID19 symptoms subsided. After 8 days of treatment her sense of smell started to regain slowly. She had full sense of smell back slowly over the following 5 days.

Other data collated from her daily reports include:
Temperatures were normal for her within 4 days of symptoms starting
O₂ levels were not reported week 1. Thereafter, they are stable and normal.
Her AM fatigue improved by 43% by the start of week 2. PM fatigue improved by 69% by the end of week 2.
She regained 100% of AM muscle use from severe pain by day 4 and 100% of PM use by day 5 and had no more pain at those times.
Cough with COVID symptoms was only there for two days.
One week after COVID symptoms stopped, her new cough persisted to the end of the study (further 14 days).
No runny nose. No headaches. No changes to hearing.
In week 2 her eyes were 17% more sore in the PM than the AM and by week 314% more sore in the PM than the AM.
She had soft bowel movements in the PM for the entire 4 weeks. The AM were soft until end of Week 3. By the start of week 4 in the AM only, she had normal bowel movements.
Average nightly sleep with COVID symptoms was 7.8 hours/night increasing to 8.8 hours by week 2 when she had the new cough symptoms, and then reduced to 8.1 hours by week 4.

### Fibromyalgia Case Study (2,289,019)

A 69 year old female patient with fibromyalgia was treated with medium setting at 15 Hz.

Room temperature 23 degrees. Humidity 75%. Patient used the apparatus at her bedside.

Blood O₂ levels average 92, which is low. Pulse rate averages 62. Morning fatigue levels improved by 43% using the device as opposed to not using it. Brain fog improved both AM and PM in using the device to not using it. Aches and pain improved by 61% in the PM compared to not using the device. Aches and pain improved by 23% in the AM compared to not using the device.

### Lung Disease—Silicosis Study (2,288,997)

A patient with silicosis diagnosed 1981 (>40 years ago) was treated using the apparatus. The patient is a 78 year old female weighing 74 kg. The patient is a non-smoker and does not consume alcohol, is rarely hospitalized and not working. Rides a bike for exercise each day.

The patient was treated using the device placed at 80 cm to 1 meter from her pillow. Sleeps 8 hours/night. Considers herself fit and healthy. Room temperature 22 degrees humidity 75-80%.

Patient has had heart problems including (heart failure). Has multiple allergies, shortness of breath, sciatica verve problems. Hypertension (high blood pressure). Heart disease following 2012 stroke and heart attack. Benign paroxysmal vertigo since 2010 then had a stomach bleed in 2017. No pacemaker.

Patient was sent oximeter to measure blood O₂ and pulse rate, she had a peak flow meter to use. The device is placed 50 cm from the patient when she reads at night for up to two hours, then moved to 1 meter away for the duration of her sleep.

The patient's bedroom is above average humidity, so we expect this will slow the species slightly in their path to her breathing, so placement is more critical for results. The following results are analysed from the daily report of the patient using a 6 number measure for her self-reported perceived results twice a day AM and PM also using the supplied measuring tools:
Oxygen levels are stable AM and PM across the study at a level of 97.
Peak flow meter measurements decreased in the AM compared to the PM for the first 2 weeks and then improved by week 3 and 4.
Fatigue levels were worst the week not using the apparatus, and slowly improved thereafter. The PM fatigue levels improved by 45% from Week 1 to Week 4. The AM fatigue levels improved by 92% from Week 1 to Week 4.
Shortness of breath, coughing and mucous production were all worst the week not using the device, and deteriorated further by the end of week 1. It took until weeks 3 and 4 to recover. AM: Shortness of breath improved by 50%, cough improved 10% and Mucous production reduced, so improved by 64%.

### Lyme Disease Study (2,289,002)

A patient with Borelia Lyme Disease Spirochaete a slow growing bacterium usually caused by a tick bite (6-36 months before symptoms) with multisystem illness and multisystem breakdowns. Doctor has had her on antibiotics as well. Medications: Minomycin 100 mg/day, Natural thyroid extract 120 mg/day.

The patient is a 65 year old female weighing 64.5 kg and of medium frame. Good diet Does not eat cheese, eggs or red meat. Non-smoker. Small amount of exercise per day. Sleeps 8 hours per night. Had total thyroidectomy in 2010 to remove goiter.

The patient has ulcerated, inflamed sores and a number of formed scars mainly on face, back and legs. Rash called erythema migrans. Fever, headaches, itch, fatigue doctor told her it can infect internal organs.

The patient reports better results by using two apparatus at once on her legs and back instead of one apparatus. Skin has improved within four weeks alleviating the pain of lyme disease but still quite scarred. She reported the apparatus is helping her focus on relaxing and assisting her to sleep better in the interim, case study continues.

### Autoimmune Disease—Transverse Myelitis (2,289,022)

A patient with Transverse Myelitis was treated using this apparatus. The patient is a 54 year old male, non-smoker. Severe transverse myelitis—inflammation of the spinal chord. Patient was first diagnosed 40 years ago, tried many solutions and had no results. As a result, suffers depression, obesity and chronic fatigue.

The patient cannot walk without severe pain of back and legs. Leg is dragged and muscles of back, hips and legs have become contorted. Uses walker and/or wheelchair if he's mobile at all, spends most days in bed. Original settings were medium output and 32 Hz however it became apparent we needed to focus on helping stabilize his mood first, so we changed him to a medium-strong setting and 25 Hz after the first two weeks to manage his depression. We then used placement variations instead of altering settings to manage further intense treatments as we monitored him closely. These ranged from 20 cm to one meter.

After the first 30 days he reported feeling 2-5% better, some days better than others. After 12 weeks, he reported ‘incredible results and relief from his leg pain, since using the morning 15-minute intense treatment’ (20 cm). 4 weeks later he is well enough to attempt working again and started an office job in sales after many years of being bed bound and unemployed. Mood is considerably improved. Now we turn focus to his nervous system as his ability to walk slowly improves. Patient did a video of himself showing his excitement at starting to walk again, albeit slowly and truncated. Treatment and monitoring continues.

### Autoimmune Disease—Lupus & Diabetes Type 2 (2,288,990)

A 44 year old female patient diagnosed with Lupus and has Hashimoto's, Diabetes (Type 2), Connective tissue disease was treated using the apparatus. Non-drinker, non-smoker. Low carb and low sugar diet. Patient has multiple typical auto-immune symptoms including daily severe headaches starting every morning, constant muscle pains all over the body, brain fog, extreme chronic fatigue, daily nausea, continuous skin rash on face, sensitivity to sunlight. Patient stated sensitivity to sun light is a side effect of one of her drugs.

Within the first three days of use of the apparatus at one metre from the patient in bed as she sleeps, she wakes with no more severe headaches in the morning and her AM blood O₂ levels improved by day 2. The one day she did not use the device, her regular morning headaches returned. Feels more energetic in the mornings after the first couple of days. Less muscular pain than previously experienced by week 2.

Measured blood oxygen levels are now stable. By weeks 3-4 all symptoms had reduced by 50% and kept reducing until week 7 when all symptoms had disappeared with the exception of: 1. a low-level headache once per fortnight and 2. the severity of the skin rashes which had reduced by 75% from the original skin rashes. In all cases, each symptom has improved by the morning from the evening readings, except for the skin rashes which are slowly improving. Her markers for diabetes type 2 has now improved so by week 30 her doctor has officially cleared her of having diabetes type 2. She now lives a normal life.

### Microbial Growth Following Negative Ion Exposure (UZZA00018)

Nasal and upper respiratory tract swabs were obtained and immersed into 1 mL sterile saline solution before being vortexed for one minute. Following that, a sterile inoculation loop was used to spread the liquid containing the bacteria onto ten Mueller-Hinton agar (MHA) plates—five plates to act as the control for the experiment with no exposure to the air species and energy fields and five plates for the apparatus treatment using the above-described apparatus. In total, there were four nasal and six upper respiratory tract MHA plates. The respiratory mucosal sample was obtained from a 19-year-old female with a current upper respiratory tract illness, suspected to be “strep throat”. The nasal sample was extracted from a 21-year-old female without any signs of illness.

The MHC plates were left to grow for three hours in a controlled environment, with the experimental group being exposed to a constant flow of the air species from the device and the control group being exposed to normal ambient air flow. During the intervention, the concentration was regularly monitored. Following that, the plates were incubated at 37° C. in a specialized incubator used for microbial cultures. The plates were inverted to prevent the condensation droplets of falling on the surface of the agar. The PhD's monitored the plates every 6-12 hours for a period of five days. The colonies present on the plates were inspected, analyzed and photographed.

Several large colonies developed on the MHC plates over the course of five days. At the completion of the experiment, colonies were examined and counted. The number of the colonies is indicative of the rate of growth of the bacteria in the MHC plates. There was a reduction in the average count of colonies on the MHC plates after the ‘three hours’ of exposure to the air species and energy fields in comparison to the control plates by an average of 72%. There was also a visible difference in the number of the colonies between the two groups. This is consistent with the numerical results.

The three-hour exposure to air species and energy fields of the apparatus appeared to have an inhibitory effect on the microbial growth for both upper respiratory tract and nasal cavity colonies present on the MHC plates. These preliminary findings support the inhuman results being experienced to date and the exploration of more research.

### Virus Long Haul, Allergy Multi-System Conditions (2,289,010)

72 year old female suffering multi-system conditions from a virus laden mosquito bite 38 years ago which put her into a coma and she's suffered more and more since. 85 kg, non-smoker. She has multiple allergies, immune system weakness, severe sensitivities to EMF, foods, chemicals and weather changes. Respiratory conditions, fibromyalgia, type 2 diabetes, hay fever, chronic nasal drip and osteoarthritis.

Given patient is a long-haul viral sufferer of almost 40 years settings are to commence for strong allergy's but with a weak frequency of 3-4 Hz. Placement to commence at 60 cm for first month cascading an extra 5 cm each month up to a 1 mtr distance from her center pillow. Patient to provide a one week base line of her data and observational records.

After 10 days of use patient had her first sleep through the night in many years. Oxygen levels took 4 weeks to stabilize and she is breathing easier. Hay fever is gone by week 4, reporting more energy. Nasal drip stopped in week 9. Ongoing treatment

### Sleep Apnea & PTSD (2,289,013)

65 year old male diagnosed with sleep apnoea 8 years ago and is a major sufferer of PTSD. Has mild asthma, chronic insomnia, brain fog. Used to suffer pericarditis and anemia and has a course of radiation 23 years ago from leiomyosarcoma. Taking 3-5 mg Imovane infrequently and 3 mg Naltrexone daily.

Patient felt worse on the commercial sleep apnea machine so he sold it a couple of years ago. Patient's pulse and blood O₂ is average and improved by the second week to normal and is stable as did his fatigue levels. By the third week he was able to sleep through the night 5 out of 7 nights, for the first time in many years.

Patient commented he can think clearer and that his brain fog and low-grade persistent headache has now gone. He wakes feeling ‘groggy’ but that is improving. His mood has improved and his wife comments he doesn't snore much anymore.

### Case Study Overview

COPD and sleep apnoea: oxygen improves by 34.3% (2,288,992);

Sleep apnoea & PTSD: all symptoms improved (2,289,013);

Respiratory pathogens: are eliminated by 72% in the first 3 hours or use; (VZZA00018);

Lung disease (Silicosis): improved breathing, fatigue and mucous production by week4 (2,288,997);

Asthma: shortness of breath improves by 48% (2,289,000);

Asthma: daily cough gone no longer on steroids reduced Ventolin (2,288,993);

Emphysema: fatigue, cough and mucous improves (2,289,006);

COVID-19: patients treated in Wuhan, none deteriorated (2,289,015);

COVID-19 anosmia: sense of smell returns by day 18 and symptoms resolved by day 35 (2,289,018);

Glioblastoma/Lung cancer terminal: palliative care improved O₂ now in remission (2,288,996);

Glioblastoma terminal: palliative care improved in remission (2,289,005);

Breast cancer 5cm deep tumor eliminated through skin and into margins (2,288,998);

Basal cell carcinoma of the ear: amputation no longer required (2,288,991);

Surgical wound: heals faster, less scarring and reduced color (2,288,995);

Diabetic foot wound: amputation no longer required (2,288,994);

Severe eczema: resolved in 40 days (2,289,001);

Fibromyalgia: pain and breathing improved by day 30 (2,289,019);

Phagocytes: shows responses to apparatus species and energy (VZZA00015);

Auto-Immune disease—Lyme disease: pain resolved in 30 days (2,289,002);

Auto-Immune disease—Lupus, diabetes: Week 7 most symptoms gone. By week 30, no longer has markers for diabetes 2 (2,288,990);

Auto-Immune disease—Transverse myelitis: out of wheelchair and now working (2,289,022)

Severe long haul viral & allergies: combination disease states no longer in pain with resolved hay fever and energy by week 6 ongoing (2,289,010);

Accordingly, the above-described arrangements provide an apparatus and system for producing corona cold air plasma (CAP) reactive oxygen and nitrogen species (RONS) and other quantum air species with a negative and/or neutral polarity through pulsed electromagnetic fields (PEMF) from ambient air temperature and non-equilibrium atmospheric air pressure. In one example, the purpose is to ‘generate various short and long lived reactive and non-reactive quantum air species, such as electrons, ions, atoms, molecules, radicals, excited states and in this case ions of negative and neutral polarity (Air Species’) through quantum PEMF portals using a custom designed circuit which ‘pushes’ and drives the Air Species into the organism from the apparatus through the electrical circuit. This process can create a gentle ionic wind at the needles of the device as the air species travel to and into the body at high speed whilst spinning fast, they are atomically hot but cold to touch. The ions charge is then bound to this vibrating and spinning molecule as it is driven by the PEW' energy into the organism where the subsequent current flow causes molecular collisions, vibrational resonances in the fluid channels throughout the body and resulting positrons and photons of light which are energised from the cold fusion reactions inside the organism to then create further reactions such has H₂ and molecules but these molecules do not go beyond the organism. The electrical current causes a magnetic field which intersects at right angles to interact in and/or on the subject and an internal physiological collision environment is formed as the repulsive and attractive pressures cause the initiation of the ‘life force’ otherwise known as dark energy/aurora/shekinah/Gods breath/Spirit, which we choose to name (‘Ruach life force’). This life force is the same force field which holds together the atoms inside a molecule. Inside the organism of the human body it energizes the Biofields and fluid currents as it creates photons and light from the collisions initiating molecular and systemic actions and reactions to take place. The novel high-speed current generated by the apparatus can severely pull and rip the electron off the molecule, so it is instantly detached and sent back into the electrical circuit along with other quantum energy species like neutrinos. They move right through and out of the body as a conductive charge, whilst the molecule is both retained and energized inside the body along with its fluids and biofield cavities now excited from the presence of the Life force field ‘Ruach’.

In one example, current flows through the organism because it urgently seeks the ground path to complete the circuit. This push of the electrical fields and pull of the right-angle magnetic fields from the initial negative ions generated through the CAP RONS, creates the internal Ruach life force which leaves biological impacts at magnetic speeds as the circuit energy needs to pull the electrons back into the circuit (magnetic speed=the speed of light). The circuit concurrently ‘pulls’ the spare electrons and neutrinos straight back into the apparatus to find its ground potential being an accessible grounding ring inside the apparatus, thereby completing the circuit for travel through the common conductively grounded organism and the grounding ring. (‘Energy Species’)

In one example, a grounding ring inside the device completes the circuit at the end after the impact through the organism. The energy species finds this grounding ring via a novel isolation bridge modification inside the AC mains adaptor which forces the electrons to find their way back to the apparatus for ground, where they are then raised up to high potential and ejected back into the air again to form more Air Species via the needle assembly. These cannot be seen or measured but we know of their existence because we can see their behavior and the trail they leave behind.

The above-described arrangements can also provide a method of using this system fora total biological and therapeutic rebalancing solution, being a ‘therapeutic Systemic Selective Adaptogen’ (‘SSA’). Application of these air and energy species is through the human respiratory system and through the skin for transdermal absorption using in part electroporation, plasma-poration and other methods.

The complexity and uniqueness of this mix of multidisciplinary sciences creates a technology with new science which produces a large range of ‘systemic selective adaptogen’ biologically active and positive responses in the organism's body and blood across most health ailments. In this regard, the response is selective (for example, causes apoptosis whilst creating immunomodulating responses), it is systemic because it can cross the blood brain barrier and impact the central nervous system using retrograde nerve transport through the vagus nerve (respiratory) and impact every system of the human body with masses of active mechanisms far too many to put in this patent but will become multitudes of patents under this one. Additionally, it is an adaptogen, which is a novel mix of the life force, the PEMF and the air species and polarities, which mean they together help the body adapt where it needs it most naturally, using its own mechanisms all evidenced by the results from my testing.

What appears to be a lack of specificity for treatment conditions, may provoke scepticism however multiple, non-specific effects are common and recognised as the target system(s) of treatments, govern the activities and functions of many different aspects of the organism such as hormonal, central nervous system, immune system and the like which respond safely to the captured forms of the Air Species, Energy and Ruach life force.

The above-described arrangements can be applied to the fields of global public health, medicine, biology, agriculture, materials, environmental sciences, food production, water and beverages including like processes of activating, purifying, preventing and treating gases, liquids and solids and in new emerging areas of science and technology including astrophysics.

Further features of the above-described apparatus will now be described.

Plasma is a unique fourth state of matter behind solids, liquids and gases in size. Plasma physics air species and waves are the smallest and are not naturally available on earth. Humans can see this ‘physics’ plasma above the arctic ocean called the ‘polar aurora or the northern lights’ colored green/blue clouds in the sky; which is a visible plasma created from the collision of charged particles (e.g., electrons) in the magnetosphere (100-200 km above earth) where atoms and atmospheric gases in magnetic fields collide causing the atoms to emit light and mostly atomic oxygen (green color) (as this technology produces) or atomic and molecular nitrogen (blue and purple color). CAP creates these quantum air species including Reactive Oxygen and Nitrogen Species (RONS) and Reactive Oxygen Species (ROS).

CAP is made up of various components electrons, ions, atoms, molecules, radicals, excited states, in this patent the ‘air species’ generated inside the CAP are either of a negative or neutral polarity. With the PEMF these energetic electrons can produce charged species, radicals, excited states and photons inside the ‘plasma plume’ at the needle tips efficiently. As the species are small and spinning at very high speed through non-equilibrium ambient pressure they accelerate using the PEMF through the portals and deliver fluxes of negative ions into and onto the organism. These enable surface modification of skin and mucous membranes by inpart but not limited too etching, plasmaporation, electroporation, diffusion, activation and deposition plus more all of which are essential for molecule uptake and energizing after the photons are generated.

These processes along with the circuit of the above-described apparatus cause collisions at very high speed but at atomic size which generates light photons. For example, generating an abundance of O and O− in our case transports the photons to create O2* inside the mitochondria, where it picks up Hydrogen to then make H₂O₂ or H₂O. Light has no mass and no charge, it has a wavelength and its ability to do work in biology at the plank scale is related to the geometry of the atoms own electrons, which is how the light interacts with the body. As it is both a wave and a particle but has no mass or charge, the geometry of the electric field and magnetic field create the intersection of fields at right angles to each other creating the Fenton reactions.

There are many highly selective pathways that the body generates when it is inspired to function correctly. For example, species such as NO have a healing effect from the CAP in the right proportion and frequency, and species like O have a killing effect from the CAP in the right proportion and frequency. The body's own ability to balance these effects in its adaptive and innate manner means the human body is capable of balancing what it needs for the individual on its own, once it is in the right environment with the right balance of species and energy is introduced. This is why this technology is adaptive.

There are various types of CAP medical devices in development, for example DBD or Jet (N-APPJ) plasmas, there are no other CAP medical devices in the world which function across all the body's systems as a Systemic Selective Adaptogen. Most require man made mixes of noble gases and are to be used only by a medical practitioner due also to their potency and high frequencies mostly in the fields of wound care, dental and cancer. Plasma sciences is a multi-disciplinary field comprising of for example fluid dynamics, physics, thermodynamics, atomic and molecular physics, mathematics, neuro-quantology, nanotechnology, quantum physics, chemistry, electrodynamics, electrical engineering, medicine, biology, epigenetics, neurology to name just a few of the 37 identified with this technology.

RONS/ROS are fundamentally critical signaling molecules, combined with the right current, energy and frequency amongst other variables they will travel to and/or influence all cells and all liquid cavities throughout the organism to enable proper functions and trigger such a large amount of quantum phenomena that it would take us a lifetime to research and understand each one. The mechanisms of action are numerous and the results are across all systems of the body, such as enhancing phagocytosis, balancing hormones, insulin, accelerating the proliferation of fibroblasts but not limited to only this. The hydroxyl radical OH for example which commences at the point of the plasma species meeting the mucous membrane fluids (due to the PEMF produces peroxidation of unsaturated fatty acids of the lipids of the cell membranes. Also the long lived species nitric oxide (NO) which is known to regulate the immune system, collagen synthesis and proliferation of keratinocytes.

Due to the stressors of modern life, pollutants in air, water and the food chain including our plethora of frequency waves in our environmental air across the entire earth surfaces, we have imbalanced biological systems; this affects all of humanity, plants and animals because we are all electrical beings made of 70% water or more. This imbalance leads to an inability to fight infection, virus, bacteria and other pathogens, increase in illness and disease and an inability to respond to good pharmaceuticals and vaccines as we should. This device provides a safe quantity and delivery mechanism for these RONS/ROS into the human body, central nervous system (CNS) and blood, which treat all systems of the body at once and over time. It improves the health of any individual exposed to it. One cannot overdose on it and it works alongside pharmaceuticals and vaccines without harm; for all ages and conditions of humanity whilst providing a solution for pharmaceuticals and vaccines to work as intended.

The above-described apparatus can generate cold air plasma before the negative ions in which short and long lived RONS/ROS and other quantum air species are created inside the cold air plasma plume and are also biologically active up to two meters (not just 2-5 mm as per existing CAP industry) where normally only long-lived species benefits would be seen

The above-described apparatus can produce corona CAP from ambient air and does not require gas tanks to drive its air energy. It uses negative and neutral polarity only others don't.

The above-described apparatus can combine extremely weak frequencies with PEMF, a weak CAP and a magnetically strong electrical circuit leaving weak currents to achieve repeatable biological results because the metabolism of all living organisms is accompanied by charge transport and known redox and photosynthesis processes.

Inside the human body the pushed PEMF and the pull of the circuit grounding ring create an intersection collision where magnetic fields are created at right angles to facilitate formation of Ruach life force which affects the fluidic, biofields and biological currents of the human body as its resulting photons cause molecular reactions and the combined currents removal of the electron from the molecule, leaving the molecule behind the electron travels back to the circuit and back into the grounding ring inside the device facilitated by the AC adaptor special isolation bridge design.

The PEMF consists of various waves including but not limited to static electricity, carrier waves, Fourier waves and frequency tones (sound) matching and species correction waves to keep the spin and momentum “inside the custom made portal” towards the human air-mucous interchange these PEMFs in part along with the Ruach life force create resonant effects throughout the human body's fluids, biofields and cellular structures leading to successful cellular communications. Because the fields are weak the effects are strong.

In one example, the AC Adaptor bridge resistor allows a return path for the quantum species and energy which allows the grounded human body to be pulled into the centre of the electrical circuit so the species from the device flying back to the device are travelling at magnetic speed and a push and pull mechanism with and from the human body facilitating the smallest and fastest quantum species and energy's generated are biologically active throughout all human systems

The resulting current causes the ripping effect of removing the spare electron from the molecule to return the electron into the circuit to reach the grounding ring of the apparatus for its energy return path in the circuit, whilst the charged, reacting and energised molecule does not move outside the body.

The quantum and CAP species combined with the PEMF can cause vibrational resonances inside the respiratory system leading to retrograde transport of the air species and energy species at the same time along the vagus nerve of the mouth and nose in the upper respiratory system which allow the crossing of the blood/brain barrier to create the ongoing systemic effects throughout the entire body originating from the then Central Nervous System (CNS), where the CNS co-ordinates its own natural adaptive and innate immune responses to balance the effects of the CAP in a selective systemic and adaptive manner.

The human cells and fluids can communicate through a sound vibration resonance. A cells life is controlled by its energy frequencies which specify this language and its physical environment not by its genes, the cells awareness of this environment sets in motion the mechanisms of action that drive life. The apparatus controls this energy language and the physical environment for the cells to respond as they were intended too, and so the genetics of the organism can function as they should.

The body can absorb and respond to the air species, PEMF and Ruach life force produced through all systems of the body in this specially designed safe mix for example but not limited to the nervous system, immune system, respiratory, cardiovascular and so on with multiple benefits to assist pharmaceutical and vaccines by making this technology and science a ‘systemic selective adaptogen’.

Thus, the above-described device functions by generating a corona cold air plasma medicine RONS and other quantum air species at the needle tips of the apparatus. PEMF and other Quantum physics waves and portals are established using selectable output and frequencies. Cold air plasma species with a negative and/or neutral polarity are transferred to the subject's body by virtue of the subject's incorporation into the circuit of the device through the ground return path. Muons and neutrinos that leave the human body into the second half of the circuit from the pulling magnetic effects, as the electrons are ripped off the molecules inside the human body, generate photon light in the body, which is the life force field referred to as Ruach energy, returning back into the device. This leads to a Systemic Selective Adaptogen response for the human body, leading to the medical benefits outlined above.

Throughout the above the term subject can include living subjects, such as humans, animals, or plants.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

1. An apparatus for generating cold air plasma, comprising:
a) a housing including a plurality of spaced openings;
b) a plurality of needles, each needle being positioned in a respective opening; and
c) a driver circuit that generates an electrical signal that is applied to the needles to generate a pulsed electromagnetic field (PEMF), wherein the electrical signal is alternating at a carrier frequency and is modulated at a pulse frequency to thereby generate cold air plasma.

2. The apparatus according to claim 1, wherein the electrical signal and PEMF have varying frequency, intensity, and multiple waveforms including at least one of:
a) a non-square waveform,
b) a right-angled waveform,
c) a square waveform,
d) a sine waveform,
e) a triangular waveform,
f) complex waveforms,
g) Fourier modified waveforms,
h) a sawtooth waveform,
i) particle-wave duality, or
j) a sharks tooth waveform.

3. The apparatus according to claim 1, wherein the carrier frequency is at least one of:
a) in the range 17 kHz to 18 kHz,
b) in the range 17.25 kHz to 17.75 kHz,
c) in the range 17.4 kHz to 17.6 kHz, or
d) about 17.5 kHz±0.25%.

4. The apparatus according to claim 1, wherein the pulse frequency is at least one of:
a) in the range 0.1 Hz to 40 Hz,
b) in the range 0.1 Hz to 4 Hz,
c) less than 1 Hz,
d) about 4 Hz,
e) about 7.83 Hz,
f) about 8 Hz,
g) about 10 Hz,
h) about 16 Hz,
i) about 25 Hz,
j) about 32 Hz, or
k) about 40 Hz.

5. The apparatus according to claim 1, wherein the apparatus includes a user input that allows selection of a pulse frequency and strength.

6. The apparatus according to claim 1, wherein the driver circuit includes:
a) a rectifier configured to be attached to a main power supply,
b) a switched mode power supply coupled to the rectifier,
c) a pulse transformer coupled to the switched mode power supply, and
d) a voltage multiplier that connects the pulse transformer to the needles.

7. The apparatus according to claim 6, wherein the pulse transformer is characterized by at least one of:
a) is configured to generate an output voltage of about 3,500-4,500 volts, or
b) includes a 178:1 turns ratio.

8. The apparatus according to claim 6, wherein the electrical signal at tips of the needles has a voltage characterized by at least one of:
a) more than 12,000 volts,
b) in the range 12,000 volts to 14,000 volts,
c) in the range 12,500 volts to 13,500 volts, or
d) about 13,000 volts±0.25%.

9. The apparatus according to claim 6, wherein a power supply circuit includes a bridging resistor arrangement bridging an AC adaptor transformer, characterized by at least one of:
a) the bridging resistor arrangement provides an earth reference via a neutral line of an alternating mains power supply, or
b) the bridging resistor arrangement includes at least two resistors that provide two means of operator protection and a return path for the charged electrons from the circuit.

10. The apparatus according to claim 1, wherein the apparatus is configured to provide a return current path allowing electrons stripped from molecules and/or ions to return to the device via the subject.

11. The apparatus according to claim 1, wherein the apparatus includes an insulating member surrounding high voltage components, wherein the insulating member is characterized by at least one of:
a) has a convoluted shape; or
b) provides a current flow path to the needles.

12. The apparatus according to claim 1, wherein the apparatus includes an alternating current adaptor configured to attach to an alternating mains power supply, characterized by at least one of:
a) the alternating current adaptor includes a barrel plug that connects to a barrel socket in the housing, and wherein the barrel socket is recessed within the housing to reduce electrostatic charge build-up, or
b) the alternating current adaptor includes the pulse transformer.

13. The apparatus according to claim 1, wherein the apparatus is configured to generate a corona cold air plasma and other quantum air species with a negative or neutral polarity at the tips of the needles and/or inside the plasma plume at the tips of the needles or just beyond the needle tips in a plume space of less than about 3 mm.

14. The apparatus according to claim 1, wherein the apparatus includes a plurality of gold coated needle assemblies in electrical contact with a voltage multiplier, each needle assembly being configured to receive a respective needle.

15. The apparatus according to claim 1, wherein the needles include:
a) a shaft configured to be attached to a needle assembly within the housing; and
b) a tip projecting into opening which is a hollow semi-circumference shaped opening.

16. The apparatus according to claim 1, wherein the needles are made of a combination of metals.

17. The apparatus according to claim 1, wherein the apparatus includes up to eight needles circumferentially spaced around a circular housing and where each four sets of needles are of equal distance from each other.

18. The apparatus according to claim 1, wherein the apparatus includes one or more caps configured to be positioned in the openings to selectively deactivate one or more of the needles.

19. The apparatus according to claim 1, wherein the apparatus is configured to generate cold air species having an ion density for at least one of:
a) more than 900,000 ions per cubic centimeter at one meter from the apparatus;
b) more than 9.9 Million ions per cubic centimeter (9.9×10⁶) at 300 mm from the apparatus;
c) more than 28 Million ions per cubic centimeter (28×10⁶) at 100 mm from the apparatus; and,
d) more than 30 Million ions per cubic centimeter (30×10⁶) at 50 mm from the apparatus.

20. The apparatus according to claim 1, wherein the apparatus is configured to generate cold air species with an ozone concentration of less than 0.04 ppm.

21. The apparatus according to claim 1, wherein the apparatus is configured to generate air species with a flow rate for at least one of:
a) more than 40.3 Trillion ions per second (40.3×10¹²) at 300 mm from the apparatus;
b) more than 50.1 Trillion ions per second (50.1×10¹²) at 100 mm from the apparatus; or
c) more than 76.8 Trillion ions per second (76.8×10¹²) at 50 mm from the apparatus.

22. A method for providing therapy to a subject, the method including generating cold air plasma using an apparatus including:
a) a housing including a plurality of spaced openings;
b) a plurality of needles, each needle being positioned in a respective opening; and,
c) a driver circuit that generates an electrical signal that is applied to the needles to generate a pulsed electromagnetic field, wherein the electrical signal is alternating at a carrier frequency and is modulated at a pulse frequency to thereby generate cold air plasma.

23. An apparatus for generating corona cold air plasma, comprising:
a) a housing including a plurality of spaced openings;
b) a plurality of needles, each needle being positioned in a respective opening;
c) a driver circuit that generates an electrical signal that is applied to the needles to generate a pulsed electromagnetic field (PEMF), wherein the electrical signal is alternating at a carrier frequency and is modulated at a pulse frequency to thereby generate corona cold air plasma RONS and other quantum species of negative and neutral polarity energized by Ruach life force, and wherein when a subject is exposed to the generated corona cold air plasma, the apparatus induces a systemic selective adaptogen response inside the subject.

24. The apparatus according to claim 23, wherein the systemic selective adaptogen response is induced and energized by Ruach life force field energy and its subsequent water electrolysis for Hydrogen (H₂) production.
